(19) United States
(12) Patent Application Publication
(10) Pub. No.: US 2023/0390341 A1
(43) Pub. Date: Dec. 7, 2023

(54) METHODS AND COMPOSITIONS FOR PREVENTION AND TREATMENT OF GRAFT VERSUS HOST DISEASE

(71) Applicant: UNIVERSITY OF MIAMI, MIAMI, FL (US)

(72) Inventors: Evangelos V. Badiavas, Coral Gables, FL (US); Krishna Komanduri, Miami, FL (US); Despina Stelios Kolonias, Miami, FL (US); Luis Rodriguez-Menocal, Miami, FL (US); Eric D. Wieder, Miami, FL (US)

(21) Appl. No.: 18/135,795

(22) Filed: Apr. 18, 2023

Related U.S. Application Data
(60) Provisional application No. 63/332,621, filed on Apr. 19, 2022.

(51) Int. Cl. A61K 35/28; A61K 45/06; A61K 9/00; A61P 37/06

(52) U.S. Cl. CPC A61K 35/28; A61K 45/06; A61K 9/0019; A61P 37/06; A61K 2035/124

(57) ABSTRACT

A pharmaceutical composition for use in preventing or treating graft versus host disease (GVHD) in a subject wherein the composition includes intact microvesicles isolated from a biological fluid using polyethylene glycol (PEG) precipitation, wherein administration of the pharmaceutical composition alleviates or prevents one or more symptoms of GVHD in the subject. Also described is a method of preventing or treating graft versus host disease (GVHD) in a subject comprising administering to the subject a pharmaceutical composition comprising intact microvesicles isolated from a biological fluid of an unrelated or related donor using polyethylene glycol (PEG) precipitation wherein one or more symptoms of GVHD comprising weight loss, cutaneous tissue damage, subcutaneous tissue damage, cutaneous inflammation, satellite cell necrosis, truncated lifespan, and/or subcutaneous inflammation are prevented or alleviated in the subject.

### RELATED APPLICATIONS

This application claims priority to U.S. Provisional Patent Application Ser. No. 63/332,621, filed Apr. 19, 2022, the entire disclosure of which is hereby incorporated herein by reference.

### BACKGROUND

Hematopoietic stem cell transplantation is a procedure utilized for many patients at high risk for or having relapsed malignant or non-malignant diseases and autoimmune diseases. The procedure involves the infusion of hematopoietic stem cells into the patient in order to reestablish blood cell production in the patient when the bone marrow or immune system of the patient is at risk. The stem cells to be infused can be donated by the patient but can be obtained from another person (allogeneic.)

Patients who receive hematopoietic stem cell transplants have significantly higher mortality rates than normal, which rates remain high for about 10 years after the transplantation (Martin P J, et al. Life expectancy in patients surviving more than 5 years after hematopoietic stem cell transplantation. J Clin Oncol. 2010; 28(6):1011-1016.) A major cause of post-transplant morbidity and mortality is graft-versus-host disease, which has acute and chronic forms and is a major cause of non-relapse mortality and decreased quality of life. In GVHD, after allogenic transplantation of stem cells from a donor (graft) into a patient (host), the donor cells view the patient's cells as foreign, and attack them, which causes tissue and organ damage in the patient. GVHD occurs in about 25-50% of patients after the receive an allogeneic transplant.

Although regimens have been developed to decrease early transplantation-related mortality, little progress has been made in preventing or decreasing later transplantation-related mortality, such as that arising from GVHD(Id.) Accordingly, there remains a need for methodologies that prevent or decrease the incidence and severity of GVHD in patients.

### SUMMARY

In one aspect, a method of preventing or treating graft versus host disease (GVHD) in a subject comprising administering to the subject a pharmaceutical composition comprising intact microvesicles isolated from a biological fluid of an unrelated or related donor using polyethylene glycol (PEG) precipitation, wherein one or more symptoms of GVHD comprising weight loss, cutaneous tissue damage, subcutaneous tissue damage, cutaneous inflammation, satellite cell necrosis, truncated lifespan, and/or subcutaneous inflammation are prevented or alleviated in the subject, is provided.

In another aspect, a pharmaceutical composition for use in preventing or treating graft versus host disease (GVHD) in a subject comprising intact microvesicles isolated from a biological fluid using polyethylene glycol (PEG) precipitation, wherein administration of the pharmaceutical composition alleviates or prevents one or more symptoms of GVHD in the subject, wherein the one or more symptoms of GVHD comprise weight loss, cutaneous tissue damage, subcutaneous tissue damage, cutaneous inflammation, satellite cell necrosis, truncated lifespan, and/or subcutaneous inflammation, is provided.

In other embodiments, the subject has GVHD as a result of receiving a hematopoietic stem cell transplant or a bone marrow transplant. In some embodiments, the subject has received a hematopoietic stem cell transplant from a person wherein the donor is matched, partially mismatched, or completely mismatched. In some embodiments, the hematopoietic stem cells can be sourced from bone marrow, peripheral blood and/or umbilical cord blood, which is freshly collected and/or cryopreserved then infused.

In some embodiments, the isolated microvesicles are administered in combination with an additional therapeutic agent for the treatment of GVHD. In some embodiments, the additional therapeutic agent is selected from the group consisting of a steroid, anti-metabolite, calcineurin inhibitor, mTOR inhibitor, kinase inhibitor, signal transducer and activator of transcription (STAT) inhibitor, and nucleotide analog inhibitor. In some embodiments, the additional therapeutic agent or process is selected from the group consisting of tacrolimus, monoclonal and/or polyclonal antibodies including antithmyocyte, globulin, abatacept, sirolimus, post-transplant cyclophosphamide, itacitinib, ibrutinib, belumosudil, and extracorporeal photopheresis. In still other embodiments, treatment of the subject with the isolated microvesicles lowers the required dose of the additional therapeutic agent for treating GVHD.

In certain exemplary embodiments, the isolated microvesicles comprise one or more of the following: exosomes, apoptotic bodies, ectosomes, nanovesicles, microparticles, membrane particles, extracellular vesicles, and shedding vesicles.

In some exemplary embodiments, when administration of the pharmaceutical composition is terminated, the subject survives for a period of time without further administration of the pharmaceutical composition, and wherein the period of time where the subject survives is more than ninety days after discontinuing treatment. In some embodiments, the normal regulatory mechanisms of the subject are preserved.

In some exemplary embodiments, the colon surface area of the subject appears closer to a normal/non-GVHD phenotype after administration of the pharmaceutical composition and the subject maintains weight and vigor.

In some exemplary embodiments, the pharmaceutical composition is administered to the subject either at the time of transplant, before the transplant, after the transplant, or a combination thereof.

In certain exemplary embodiments, the biological fluid is from mammalian cells. In some embodiments, the mammalian cells are human cells. In other embodiments, the biological fluid contains stem cells. In exemplary embodiments, the stem cells are mesenchymal stem cells. In certain embodiments, the stem cells are derived from bone marrow. In additional embodiments, the stem cells are allogeneic in origin.

In some embodiments, the isolated microvesicles are precipitated from the biological fluid using polyethylene glycol (PEG). In some embodiments, the PEG comprises a weight of about 6000-20000 Da.

In other embodiments, the isolated microvesicles are purified using tangential flow filtration.

In certain exemplary embodiments, the isolated microvesicles range in size from 2 nm to 5000 nm. In some embodiments, the isolated microvesicles range in size from 100 nm to 800 nm.

In another embodiment, the isolated microvesicles have a molecular weight of at least 100 kDa.

In still other embodiments, the GVHD is acute GVHD. In some embodiments, the GVHD is chronic. In some embodiments, the GVHD is refractory to a treatment selected from the group consisting of a steroid, anti-metabolite, calcineurin inhibitor, mTOR inhibitor, kinase inhibitor, signal transducer and activator of transcription (STAT) inhibitor, and nucleotide analog inhibitor.

In certain exemplary embodiments, the intact microvesicles deliver one or more bioactive agents comprising check-point inhibitors, transcription factors, peptides, subcellular organelles, and/or nucleic acids to the subject.

In other exemplary embodiments, the isolated microvesicles increase the number of regulatory T cells (Tregs) in the blood and/or target tissue of the subject. In some embodiments, the Tregs are FOXP3+.

In certain exemplary embodiments, the isolated microvesicles are delivered to the subject by systemic administration, local injection, and/or topically to skin or eye. In some embodiments, the intact microvesicles are delivered intravenously to the subject.

### BRIEF DESCRIPTION OF THE DRAWINGS

The accompanying drawings illustrate various embodiments of the present disclosure.

FIG. 1 shows one embodiment of a microvesicle isolation method.

FIG. 2 shows an alternate embodiment of a microvesicle isolation method.

FIG. 3A-D show electron micrographs of microvesicles derived from medium conditioned using human bone marrow-derived mesenchymal stem cells isolated by the ultracentrifuge method described (FIGS. 3A and 3B) and isolated according to the methods of the present disclosure (FIGS. 3C and 3D) at the magnifications shown in the panels.

FIG. 4A-D show electron micrographs of microvesicles derived from medium conditioned using porcine bone marrow-derived mesenchymal stem cells isolated by the ultracentrifuge method (FIGS. 4A and 4B) and isolated according to the methods of the present disclosure (FIGS. 4C and 4D) at the magnifications shown in the panels.

FIG. 5A-D show electron micrographs of microvesicles derived from medium conditioned using murine bone marrow-derived mesenchymal stem cells isolated by the ultracentrifuge method (FIGS. 5A and 5B) and isolated according to the methods of the present disclosure (FIG. 5C and FIG. 5D) at the magnifications shown in the panels.

FIG. 6A-C show electron micrographs of microvesicles isolated from human plasma. FIG. 6A-C show the microvesicles under increasing magnification, as shown by the scale bars in the panels.

FIG. 7A-C show electron micrographs of microvesicles isolated from porcine plasma. FIG. 7A-C show the microvesicles under increasing magnification, as shown by the scale bars in the panels.

FIG. 8A-C show electron micrographs of microvesicles isolated from human urine. FIG. 8A-C show the microvesicles under increasing magnification, as shown by the scale bars in the panels.

FIG. 9 shows a Western blot, reporting the expression of HSP70, CD63, STAT 3 and phosphorylated STAT3 in lysates of human bone marrow-derived mesenchymal stem cells, microvesicles isolated from medium conditioned using human bone marrow-derived stem cells, prepared by ultracentrifugation (hMSC MV Ultracentrifuge), or the methods of the present disclosure, as described in Example 2 (hMSC PEG Precipitation). Microvesicles derived from human plasma and human urine, as described in Example 2 were also analyzed. (Human plasma PEG Precipitation) and (human urine PEG Precipitation) respectively.

FIG. 10A-D shows the uptake of the microvesicles isolated from culture medium conditioned using bone marrow-derived stem cells obtained from a green fluorescent protein (GFP) expressing mouse into human dermal fibroblasts. Cell nuclei, resolved using Hoechst 33342 dye, are shown in the panel labeled “Hoechst33342” (FIG. 10A). Cells, resolved using vybrant dye, are shown in the panel labeled “Vybrant-Dio” (FIG. 10B). GFP-labeled microvesicles are shown in the panel labeled “GFP” (FIG. 10C). A panel where images obtained from all three dyes are overlaid is seen in the panel labeled “Composite” (FIG. 10D).

FIG. 11A-D shows the uptake of the microvesicles isolated from culture medium conditioned using bone marrow-derived stem cells obtained from a GFP expressing mouse into human dermal fibroblasts. Cell nuclei, resolved using Hoechst 33342 dye, are shown in the panel labeled “Hoechst33342” (FIG. 11A). Cells, resolved using vybrant dye, are shown in the panel labeled “Vybrant-Dio” (FIG. 11B). GFP-labeled microvesicles are shown in the panel labeled “GFP” (FIG. 11C). A panel where images obtained from all three dyes are overlaid is seen in the panel labeled “Composite” (FIG. 11D).

FIG. 12 illustrates a GVHD negative control experiment using C57BL/6 mice that receive T cell depleted (TCD) bone marrow transplant only. These mice were symptomatic, but ultimately survived.

FIG. 13 illustrates an acute GVHD positive control experiment using C57BL/6 mice without EV treatment. C57BL/6 mice received bone marrow and splenic T cells. As a complete MHC-mismatch model designed for 100% non-survival was used, as anticipated, these mice did not survive GVHD.

FIG. 14 illustrates the treatment group of acute GVHD model C57BL/6 mice and EV administration. C57BL/6 mice received bone marrow and splenic T cells. The mice also received EVs purified from the bone marrow of a DBA/2J mouse and go on to survive for a period of time until they were terminated due to the costs of prolonged housing. Of note, these mice did not succumb to their disease within the timeframe tested.

FIG. 15A-C depicts NanoSight™ nanoparticle tracking analysis of the microvesicles used in the experiments of Example 9. FIG. 15A graphically depicts finite track length adjustment (FTLA) concentration versus size of the microvesicles. FIG. 15B graphically depicts averaged finite track length adjustment (FTLA) concentration versus size of the microvesicles. FIG. 15C graphically depicts intensity (a.u.) versus size of the microvesicles.

FIG. 16 depicts a schema of experiments 1-4 of example 9 that studied exosome treatment in a major MHC-mismatch mouse model of GVHD.

FIG. 17A-C graphically depict the results of experiment 1 of example 9 that tested 1× and 0.1×EV doses administered 3 times per week in the treatment of GVHD in mice. The blue line depicts mice treated with bone marrow alone, the negative control group for GVHD. The red line depicts mice treated with bone marrow and donor T cells, the positive control group for lethal GVHD. The green line depicts mice treated with the 1× dose of EVs. The purple line depicts mice treated the 0.1× dose of EVs. FIG. 17A depicts percent body weight of the mice versus days post-transplant. FIG. 17B depicts total clinical score of the mice versus days post-transplant. FIG. 17C depicts percent survival of the mice versus days post-transplant. In both EV treatment groups, there was a slight decrease in overall GVHD clinical scoring and slightly prolonged survival.

FIG. 18A-C graphically depict the results of experiment 2 of example 9 that tested 1× and 0.1×EV doses administered once per day in the treatment of GVHD in mice. The blue line depicts mice treated with bone marrow alone, the negative control group for GVHD. The red line depicts mice treated with bone marrow and donor T cells, the positive control group for lethal GVHD. The green line depicts mice treated with a 1× dose of EVs. The purple line depicts mice treated a 0.1× dose of EVs. FIG. 18A depicts percent body weight of the mice versus days post-transplant. FIG. 18B depicts total clinical score of the mice versus days post-transplant. FIG. 18C depicts percent survival of the mice versus days post-transplant. Treatment with both EV doses slightly decreased overall GVHD clinical scoring and significantly prolonged survival.

FIG. 19A-C graphically depict the results of experiment 3 of example 9 that tested 5× and 10×EV doses administered once per day in the prevention (prophylaxis) of GVHD in mice. The blue line depicts mice treated with bone marrow alone, the negative control group for GVHD. The red line depicts mice treated with bone marrow and donor T cells, the positive control group for lethal GVHD. The green line depicts mice treated with a 10× dose of EVs. The purple line depicts mice treated a 5× dose of EVs. FIG. 19A depicts percent body weight of the mice versus days post-transplant. FIG. 19B depicts total clinical score of the mice versus days post-transplant. FIG. 19C depicts percent survival of the mice versus days post-transplant. The results were significantly improved overall GVHD clinical scoring and prolonged survival in both EV treatment groups.

FIG. 20A-C graphically depict the results of experiment 4 of example 9 that retested a 10×EV dose administered once per day in the treatment of GVHD in mice. The blue line depicts mice treated with bone marrow alone, the negative control group for GVHD. The red line depicts mice treated with bone marrow and donor T cells, the positive control group for lethal GVHD. The green line depicts mice treated with a 10× dose of EVs. FIG. 20A depicts percent body weight of the mice versus days post-transplant. FIG. 20B depicts total clinical score of the mice versus days post-transplant. FIG. 20C depicts percent survival of the mice versus days post-transplant. The results were that daily dosing of EVs at the 10× dose significantly improved overall GVHD clinical scoring and significantly prolonged survival.

FIG. 21A-FIG. 21F depict H&E stained skin tissue samples from mice from the experiments of example 9. FIG. 21A depicts a sample from a non-GVHD control mouse 21 days after transplant. FIG. 21B depicts a sample from a GVHD model mouse with PBS vehicle 21 days after transplant. FIG. 21C depicts a sample from a GVHD model mouse with 100× dose MSC EV treatment 21 days after transplant. FIG. 21D depicts a sample from a non-GVHD control mouse 28 days after transplant. FIG. 21E depicts a sample from a GVHD model mouse with PBS vehicle 28 days after transplant. FIG. 21F depicts a sample from a GVHD model mouse with 100× dose MSC EV treatment 28 days after transplant. At Day 21 (FIG. 21B) the bracket with the star indicates the thickening of the epidermis with lymphocyte extravasation along the base of the epidermis (black arrows.) By Day 28 (FIG. 21E) the epidermis has undergone a degenerative change (above the dotted red line) with vacuolar change, acantholysis and satellite cell necrosis (black arrows.) These are findings diagnostic of acute GVHD. These changes are absent in both control mice (FIG. 21A & FIG. 21D) and extracellular vesicle treated mice (FIG. 21C & FIG. 21D). These results demonstrate that MSC-EVs attenuate cutaneous aGVHD pathology.

FIG. 22A-D depicts skin tissue samples from the experiment of example 9 in mice at day 21, these are the broader view of the day 21 samples described for FIG. 21 above. FIG. 22A depicts a sample from a non-GVHD control mouse. The epidermis and subcutaneous tissue appear normal. FIG. 22B depicts a sample from a GVHD model mouse with PBS vehicle. The yellow bracket with yellow star indicates a thickened, inflamed epidermis. The black arrow highlights an area of satellite cell necrosis within the epidermis. The red arrow demonstrates an area of inflammation and destruction of hypodermal adipose tissue. FIG. 22C depicts a sample from a GVHD model mouse with 10× dose MSC EV treatment. The epidermis appears normal. There is a mildly increased dermal inflammatory infiltrate. FIG. 22D depicts a sample from a GVHD model mouse with 10× dose MSC EV treatment. The epidermis is again normal. There is inflammation present within the hypodermal adipose tissue (red arrow) but the level of adipose tissue destruction is less than in GVHD mice treated with PBS vehicle.

FIG. 23A-I depicts tissue samples from mice at day 28 (example 9), these are the broader view of the day 28 samples described for FIG. 21 above. FIG. 23A and FIG. 23B depict samples from a non-GVHD control mouse. There is overall increased dermal, hypodermal and subcutaneous inflammation. FIG. 23B consists of only subcutaneous adipose tissue. FIG. 23C, FIG. 23D, FIG. 23E, FIG. 23F, and FIG. 23G depict samples from a GVHD model mouse with PBS vehicle. FIG. 23C illustrates focal destruction of the epidermis (black arrow) and a dense inflammatory infiltrate in the hypodermis (red arrow) with destruction of hypodermal adipose tissue. FIG. 23D also has areas of focal destruction of the epidermis (black arrows). There is also dense granulomatous inflammation in the subcutaneous adipose tissue with destruction of subcutaneous adipose tissue. This is a finding seen in the acute phase of chronic GVHD. FIG. 23E is a higher magnification of the subcutaneous adipose tissue inflammation and destruction. FIG. 23F and FIG. 23G illustrate the destruction of the epidermis with numerous necrotic keratinocytes exhibiting features of satellite cell necrosis (black arrows), a characteristic feature of acute GVHD. FIG. 23H and FIG. 23I depict samples from a GVHD model mouse with 10× dose MSC EV treatment. FIG. 23H and FIG. 23I illustrates mild chronic dermal inflammation. There is inflammation of the hypodermal fat but preservation of several areas of the adipose tissue (blue arrows). The subcutaneous fat also has some focal inflammation (red arrows) but with much of the subcutaneous adipose tissue unaffected (green arrows).

FIG. 24A-C depict H&E-stained skin tissue samples from mice at 28 days after transplant (example 9). FIG. 24A depicts a sample from a non-GVHD control mouse. FIG. 24B depicts a sample from a GVHD model mouse with PBS vehicle. FIG. 24C depicts a sample from a GVHD model mouse with 10× dose MSC EV treatment. These results demonstrate that MSC-EVs attenuate subcutaneous inflammation in aGVHD pathology (red arrows).

FIG. 25A-C depict FOXP3+ regulatory T (Treg) cells in skin tissue samples from mice at 28 days after transplant from the experiment (example 9). FIG. 25A depicts a sample from a non-GVHD control mouse. FIG. 25B depicts a sample from a GVHD model mouse with PBS vehicle. FIG. 25C depicts a sample from a GVHD model mouse with 10× dose MSC EV treatment. These results show that FOXP3+ regulatory T (Treg) cells in skin tissue that are lost in the GVHD model are restored with EV treatment.

FIG. 26A-FIG. 26C depict colon tissue samples from mice at 21 days after transplant from (example 9). FIG. 26A depicts a sample from a GVHD model mouse with PBS vehicle. FIG. 26B depicts a sample from a non-GVHD control mouse. FIG. 26C depicts a sample from a GVHD model mouse with 40× dose MSC EV treatment. As shown in here FIG. 26A, there is loss of villi and surface area in mice receiving bone marrow transplant and splenic T cells treated with vehicle (PBS). As shown in FIG. 26B, villi and surface area in mice receiving bone marrow transplant depleted of T cells alone (control) appeared mostly intact. As shown in here FIG. 26C, villi and surface area in mice receiving bone marrow transplant and splenic T cells treated with bone marrow derived extracellular vesicles appear minimally effected. These results show that EV treatment decreases the colon tissue manifestations of GVHD.

### DETAILED DESCRIPTION

Unless defined otherwise, all technical and scientific terms used herein have the same meaning as commonly understood by one of ordinary skill in the art to which this disclosure belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition.

As used herein, “graft versus host disease” or “GVHD” refers to a systemic disorder that occurs when the immune cells of a donor tissue are donated to a host, then the immune cells recognize the host tissue as foreign and attack the host's cells (Vaillant et al. Graft Versus Host Disease. [Updated 2021 Oct. 15] StatPearls Publishing; 2022 January Available from: www.ncbi.nlm.nih.gov/books/NBK538235/.) GVHD is a common complication after allogenic hematopoietic stem cell transplant (HCT) (Socié et al. Current issues in chronic graft-versus-host disease. Blood. 2014 Jul. 17; 124(3):374-84.) GVHD can be acute or chronic. Acute GVHD occurs within 100 days post-transplant and is mediated by mature effector T cells from the donor (graft) that become activated after encountering alloantigens in the recipient (host) (Whangbo et al. The role of regulatory T cells in graft-versus-host disease management. Expert Rev Hematol. 2020 February; 13(2):141-154.) Chronic GVHD occurs later than acute GVHD and is characterized by aberrant immune responses to both autoantigens and alloantigens (Id.) Chronic GVHD can also have an acute/aggressive onset with characteristic features affecting the subcutaneous tissue (often adipose tissue.)

As used herein, the expression “in combination with” means that the additional therapeutic agents are administered before, after, or concurrent with the pharmaceutical composition comprising the intact microvesicles. In some embodiments, the term “in combination with” includes sequential or concomitant administration of intact microvesicles and a second therapeutic agent. Methods to treat GVHD or an associated condition or complication comprising administration of intact microvesicles in combination with a second therapeutic agent for additive or synergistic activity, are provided.

As used herein, the expressions “microvesicles” and “extracellular vesicles (EVs)” refer to a heterogenous population of vesicles that include any of the following: exosomes, microvesicles, apoptotic bodies, ectosomes, nanovesicles, microparticles, membrane particles, extracellular vesicles, and shedding vesicles.

### Methods to Isolate the Microvesicles of the Present Disclosure

The present disclosure provides methods to isolate microvesicles (MVs), e.g., extracellular vesicles (EVs) from biological fluids without damaging the structural and/or functional integrity of the microvesicles. The present disclosure also provides methods to isolate ectosomes, microparticles, microvesicles, nanovesicles, shedding vesicles, apoptotic bodies, or membrane particles from biological fluids without damaging their structural and/or functional integrity. The present disclosure further provides MVs (e.g., EVs) and methods of using MVs (e.g., EVs) for the treatment of.

The microvesicles of the present disclosure can be isolated according to the methods described in any one of U.S. Pat. No. 10,500,231 and US 2018/0104186A1, incorporated by reference in the entirety.

As used herein, the term “microvesicles” refers to vesicles comprising lipid bilayers, formed from the plasma membrane of cells, and are heterogeneous in size, ranging from about 2 nm to about 5000 nm. The cell from which a microvesicle is formed is herein referred to as “the host cell.”

Microvesicles exhibit membrane proteins from their host cell on their membrane surface, and may also contain molecules within the microvesicle from the host cell, such as, for example, mRNA, miRNA, tRNA, RNA, DNA, lipids, proteins or infectious particles. These molecules may result from, or be, recombinant molecules introduced into the host cell. Microvesicles can also contain subcellular organelles (e.g., rough or smooth endoplasmic reticulum, golgi bodies, secretory vesicles, endosomes, lysosomes, peroxisomes, nuclear materials and mitochondria.) Microvesicles play a critical role in intercellular communication, and can act locally and distally within the body, inducing changes in cells by fusing with a target cell, introducing the molecules or subcellular organelles transported on and/or in the microvesicle to the target cell. For example, microvesicles have been implicated in anti-tumor reversal, cancer, tumor immune suppression, metastasis, tumor-stroma interactions, angiogenesis and tissue regeneration. Microvesicles may also be used to diagnose disease, as they have been shown to carry biomarkers of several diseases, including, for example, cardiac disease, HIV and leukemia.

In one embodiment, microvesicles are isolated from a biological fluid containing microvesicles in a method comprising the steps of:
a) obtaining a biological fluid containing microvesicles,
b) clarifying the biological fluid to remove cellular debris,
c) precipitating the microvesicles by adding a precipitating agent to the clarified biological fluid,
d) collecting the precipitated microvesicles and washing the material to remove the precipitating agent, and
e) suspending the washed microvesicles in a solution for storage or subsequent use.

In one embodiment, the biological fluid is clarified by centrifugation. In an alternate embodiment, the biological fluid is clarified by filtration.

In one embodiment, the precipitated microvesicles are collected by centrifugation. In an alternate embodiment, the precipitated microvesicles are collected by filtration.

In one embodiment, microvesicles are isolated from a biological fluid containing microvesicles in a method comprising the steps of:
a) obtaining a biological fluid containing microvesicles,
b) clarifying the biological fluid to remove cellular debris,
c) precipitating the microvesicles by adding a precipitating agent to the clarified biological fluid,
d) collecting the precipitated microvesicles and washing the material to remove the precipitating agent,
e) suspending the washed microvesicles in a solution, and
f) processing the microvesicles to analyze the nucleic acid, carbohydrate, lipid, small molecules and/or protein content.

In one embodiment, the biological fluid is clarified by centrifugation. In an alternate embodiment, the biological fluid is clarified by filtration.

In one embodiment, the precipitated microvesicles are collected by centrifugation. In an alternate embodiment, the precipitated microvesicles are collected by filtration.

In one embodiment, the present disclosure provides reagents and kits to isolate microvesicles from biological fluids according to the methods of the present disclosure.

The biological fluid may be peripheral blood, sera, plasma, ascites, urine, cerebrospinal fluid (CSF), sputum, saliva, bone marrow, synovial fluid, aqueous humor, amniotic fluid, cerumen, breast milk, broncheo alveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids.

The biological fluid may also be derived from the blastocyl cavity, umbilical cord blood, or maternal circulation, which may be of fetal or maternal origin. The biological fluid may also be derived from a tissue sample or biopsy.

The biological fluid may be derived from plant cells of cultures of plant cells. The biological fluid may be derived from yeast cells or cultures of yeast cells.

In one embodiment, the biological fluid is cell culture medium. In one embodiment, the cell culture medium is conditioned using tissues and/or cells prior to the isolation of microvesicles according to the methods of the present disclosure.

The term “conditioned” or “conditioned medium” refers to medium, wherein a population of cells or tissue, or combination thereof is grown, and the population of cells or tissue, or combination thereof contributes factors to the medium. In one such use, the population of cells or tissue, or combination thereof is removed from the medium, while the factors the cells produce remain. In one embodiment, the factors produced are microvesicles. Medium may be conditioned via any suitable method selected by one of ordinary skill in the art. For example, medium may be cultured according to the methods described in EP1780267A2.

In one embodiment, microvesicles are isolated from cells or tissue that have been pre-treated prior to the isolation of the microvesicles. Pretreatment may include, for example, culture in a specific medium, a medium that contains at least one additive, growth factor, medium devoid of serum, or a combination thereof. Alternatively, pretreatment may comprise contacting cells or tissues with additives (e.g., interleukin, VEGF, inducers of transcription factors, transcription factors, hormones, neurotransmitters, pharmaceutical compounds, microRNA), transforming agents (e.g., liposome, viruses, transfected agents, etc.). Alternatively, pretreatment may comprise exposing cells or tissue to altered physical conditions (e.g., hypoxia, cold shock, heat shock and the like).

In one embodiment, microvesicles are isolated from medium conditioned using cells or tissue that have been pre-treated prior to the isolation of the microvesicles. Pretreatment may include, for example, culture in a specific medium, a medium that contains at least one additive, growth factor, medium devoid of serum, or a combination thereof. Alternatively, pretreatment may comprise contacting cells or tissues with additives (e.g., interleukin, VEGF, inducers of transcription factors, transcription factors, hormones, neurotransmitters, pharmaceutical compounds, microRNA), transforming agents (e.g., liposome, viruses, transfected agents, etc.). Alternatively, pretreatment may comprise exposing cells or tissue to altered physical conditions (e.g., hypoxia, cold shock, heat shock and the like).

In one embodiment, the biological fluid is an extract from a plant. In an alternate embodiment, the biological fluid is a cell culture medium from a culture of plant cells. In an alternate embodiment, the biological fluid is yeast extract. In an alternate embodiment, the biological fluid is a cell culture medium from a culture of yeast cells.

While the methods of the present disclosure may be carried out at any temperature, one of ordinary skill in the art can readily appreciate that certain biological fluids may degrade, and such degradation is reduced if the sample is maintained at a temperature below the temperature at which the biological fluid degrades. In one embodiment, the method of the present disclosure is carried out at 4° C. In an alternate embodiment, at least one step of the method of the present disclosure is carried out at 4° C. In certain embodiments, the biological fluid may be diluted prior to being subjected to the methods of the present disclosure. Dilution may be required for viscous biological fluids, to reduce the viscosity of the sample, if the viscosity of the sample is too great to obtain an acceptable yield of microvesicles. The dilution may be a 1:2 dilution. Alternatively, the dilution may be a 1:3 dilution. Alternatively, the dilution may be a 1:4 dilution. Alternatively, the dilution may be a 1:5 dilution. Alternatively, the dilution may be a 1:6 dilution. Alternatively, the dilution may be a 1:7 dilution. Alternatively, the dilution may be a 1:8 dilution. Alternatively, the dilution may be a 1:9 dilution. Alternatively, the dilution may be a 1:10 dilution. Alternatively, the dilution may be a 1:20 dilution. Alternatively, the dilution may be a 1:30 dilution. Alternatively, the dilution may be a 1:40 dilution. Alternatively, the dilution may be a 1:50 dilution. Alternatively, the dilution may be a 1:60 dilution. Alternatively, the dilution may be a 1:70 dilution. Alternatively, the dilution may be a 1:80 dilution. Alternatively, the dilution may be a 1:90 dilution. Alternatively, the dilution may be a 1:100 dilution.

The biological fluid may be diluted with any diluent, provided the diluent does not affect the functional and/or structural integrity of the microvesicles. One of ordinary skill in the art may readily select a suitable diluent. Diluents may be, for example, phosphate buffered saline, cell culture medium, and the like.

In one embodiment, the biological fluid is clarified by the application of a centrifugal force to remove cellular debris. The centrifugal force applied to the biological fluid is sufficient to remove any cells, lysed cells, tissue debris from the biological fluid, but the centrifugal force applied is insufficient in magnitude, duration, or both, to remove the microvesicles. The biological fluid may require dilution to facilitate the clarification.

The duration and magnitude of the centrifugal force used to clarify the biological fluid may vary according to a number of factors readily appreciated by one of ordinary skill in the art, including, for example, the biological fluid, the pH of the biological fluid, the desired purity of the isolated microvesicles, the desired size of the isolated microvesicles, the desired molecular weight of the microvesicles, and the like. In one embodiment, a centrifugal force of 2000×g is applied to the biological fluid for 30 minutes.

The clarified biological fluid is contacted with a precipitation agent to precipitate the microvesicles. In one embodiment, the precipitation agent may be any agent that surrounds the microvesicles and displaces the water of solvation. Such precipitation agents may be selected from the group consisting of polyethylene glycol, dextran, and polysaccharides.

In an alternate embodiment, the precipitation agent may cause aggregation of the microvesicles.

In an alternate embodiment, the precipitation agent is selected from the group consisting of calcium ions, magnesium ions, sodium ions, ammonium ions, iron ions, organic solvents such as ammonium sulfate, and flocculating agents, such as alginate.

The clarified biological fluid is contacted with the precipitation agent for a period of time sufficient to precipitate the microvesicles. The period of time sufficient to precipitate the microvesicles may vary according to a number of factors readily appreciated by one of ordinary skill in the art, including, for example, the biological fluid, the pH of the biological fluid, the desired purity of the isolated microvesicles, the desired size of the isolated microvesicles, the desired molecular weight of the microvesicles, and the like. In one embodiment, the period of time sufficient to precipitate the microvesicles is 6 hours.

In one embodiment, the clarified biological fluid is contacted with the precipitation agent for a period of time sufficient to precipitate the microvesicles at 4° C.

The concentration of the precipitation agent used to precipitate the microvesicles from a biological fluid may vary according to a number of factors readily appreciated by one of ordinary skill in the art, including, for example, the biological fluid, the pH of the biological fluid, the desired purity of the isolated microvesicles, the desired size of the isolated microvesicles, the desired molecular weight of the microvesicles, and the like.

In one embodiment, the precipitation agent is polyethylene glycol. The molecular weight of polyethylene glycol used in the methods of the present disclosure may be from about 200 Da to about 10,000 Da. In one embodiment, the molecular weight of polyethylene glycol used in the methods of the present disclosure may be greater than 10,000 Da. In certain embodiments, the molecular weight of polyethylene glycol used in the methods of the present disclosure is 10,000 Da or 20,000 Da. The choice of molecular weight may be influenced by a variety of factors including, for example, the viscosity of the biological fluid, the desired purity of the microvesicles, the desired size of the microvesicles, the biological fluid used, and the like. In one embodiment, the molecular weight of polyethylene glycol used in the methods of the present disclosure may be from about 200 Da to about 8,000 Da, or is approximately any of 200 Da, 300 Da, 400 Da, 600 Da, 1000 Da, 1450 Da, 1500 Da, 2000 Da, 3000 Da, 3350 Da, 4000 Da, 6000 Da, 8000 Da, 10000 Da, 20000 Da or 35000 Da or any ranges or molecular weights in between.

In one embodiment, the molecular weight of polyethylene glycol used in the methods of the present disclosure is about 6000 Da.

In one embodiment, the average molecular weight of polyethylene glycol used in the methods of the present disclosure is about 8000 Da.

In one embodiment, the average molecular weight of polyethylene glycol used in the methods of the present disclosure is about 10000 Da.

In one embodiment, the average molecular weight of polyethylene glycol used in the methods of the present disclosure is about 20000 Da.

The concentration of polyethylene glycol used in the methods of the present disclosure may be from about 0.5% w/v to about 100% w/v. The concentration of polyethylene glycol used in the methods of the present disclosure may be influenced by a variety of factors including, for example, the viscosity of the biological fluid, the desired purity of the microvesicles, the desired size of the microvesicles, the biological fluid used, and the like.

In certain embodiments, the polyethylene glycol is used in the concentration of the present disclosure at a concentration between about 5% and 25% w/v. In certain embodiments, the concentration is about 5%, 6%, 7%, 8%, 9%, 10%, 11%, 12%, 13%, 14%, or 15%, or a range between any two of these values.

In one embodiment, the concentration of polyethylene glycol used in the methods of the present disclosure is about 8.5% w/v.

In one embodiment, the concentration of polyethylene glycol used in the methods of the present disclosure is about 6% w/v.

In one embodiment, polyethylene glycol having an average molecular weight of 6000 Da is used, at a concentration of 8.5% w/v. In one embodiment, the polyethylene glycol is diluted in 0.4M sodium chloride.

In one embodiment, the concentration of the polyethylene glycol used in the methods of the present disclosure is inversely proportional to the average molecular weight of the polyethylene glycol. For example, in one embodiment, polyethylene glycol having an average molecular weight of 4000 Da is used, at a concentration of 20% w/v. In an alternate embodiment, polyethylene glycol having an average molecular weight of 8000 Da is used, at a concentration of 10% w/v. In an alternate embodiment, polyethylene glycol having an average molecular weight of 20000 Da is used, at a concentration of 4% w/v.

In one embodiment, the precipitated microvesicles are collected by the application of centrifugal force. The centrifugal force is sufficient and applied for a duration sufficient to cause the microvesicles to form a pellet, but insufficient to damage the microvesicles.

The duration and magnitude of the centrifugal force used to precipitate the microvesicles from a biological fluid may vary according to a number of factors readily appreciated by one of ordinary skill in the art, including, for example, the biological fluid, the pH of the biological fluid, the desired purity of the isolated microvesicles, the desired size of the isolated microvesicles, the desired molecular weight of the microvesicles, and the like. In one embodiment, the precipitated microvesicles are collected by the application of a centrifugal force of 10000×g for 60 minutes.

The precipitated microvesicles may be washed with any liquid, provided the liquid does not affect the functional and/or structural integrity of the microvesicles. One of ordinary skill in the art may readily select a suitable liquid. Liquids may be, for example, phosphate buffered saline, cell culture medium, and the like.

In one embodiment, the washing step removes the precipitating agent. In one embodiment, the microvesicles are washed via centrifugal filtration, using a filtration device with a 100 kDa molecular weight cut off.

The isolated microvesicles may be suspended with any liquid, provided the liquid does not affect the functional and/or structural integrity of the microvesicles. One of ordinary skill in the art may readily select a suitable liquid. Liquids may be, for example, phosphate buffered saline, cell culture medium, and the like.

In one embodiment, the isolated microvesicles may be further processed. The further processing may be the isolation of a microvesicle of a specific size. Alternatively, the further processing may be the isolation of microvesicles of a particular size range. Alternatively, the further processing may be the isolation of a microvesicle of a particular molecular weight. Alternatively, the further processing may be the isolation of microvesicles of a particular molecular weight range. Alternatively, the further processing may be the isolation of a microvesicle exhibiting or containing a specific molecule.

In one embodiment, the microvesicles of the present disclosure are further processed to isolate a preparation of microvesicles having a size of about 2 nm to about 1000 nm as determined by electron microscopy. In an alternate embodiment, the microvesicles of the present disclosure are further processed to isolate a preparation of microvesicles having a size of about 2 nm to about 500 nm as determined by electron microscopy. In an alternate embodiment, the microvesicles of the present disclosure are further processed to isolate a preparation of microvesicles having a size of about 2 nm to about 400 nm as determined by electron microscopy. In an alternate embodiment, the microvesicles of the present disclosure are further processed to isolate a preparation of microvesicles having a size of about 2 nm to about 300 nm as determined by electron microscopy. In an alternate embodiment, the microvesicles of the present disclosure are further processed to isolate a preparation of microvesicles having a size of about 2 nm to about 200 nm as determined by electron microscopy. In an alternate embodiment, the microvesicles of the present disclosure are further processed to isolate a preparation of microvesicles having a size of about 2 nm to about 100 nm as determined by electron microscopy. In an alternate embodiment, the microvesicles of the present disclosure are further processed to isolate a preparation of microvesicles having a size of about 2 nm to about 50 nm as determined by electron microscopy. In an alternate embodiment, the microvesicles of the present disclosure are further processed to isolate a preparation of microvesicles having a size of about 2 nm to about 20 nm as determined by electron microscopy. In an alternate embodiment, the microvesicles of the present disclosure are further processed to isolate a preparation of microvesicles having a size of about 2 nm to about 10 nm as determined by electron microscopy.

In one embodiment, the subsequent purification is performed using a method selecting from the group consisting of immunoaffinity, HPLC, tangential flow filtration, phase separation/partitioning, and microfluidics.

In one embodiment, the isolated microvesicles are further processed to analyze the molecules exhibited on, or contained within the microvesicles. The molecules analyzed are selected from the group consisting of nucleic acid, carbohydrate, lipid, small molecules, ions, metabolites, protein, and combinations thereof.

Biological fluid comprising cell culture medium conditioned using cultured cells: In one embodiment, microvesicles are obtained from medium conditioned using cultured cells. Any cultured cell, or population of cells may be used in the methods of the present disclosure. The cells may be stem cells, primary cells, cell lines, tissue or organ explants, or any combination thereof. The cells may be allogeneic, autologous, or xenogeneic in origin.

In one embodiment, the microvesicles are obtained from mammalian cells. In an exemplary embodiment, the microvesicles are obtained from human cells.

In another embodiment, the microvesicles are obtained from stem cells. In an exemplary embodiment, the microvesicles are obtained from mesenchymal stem cells. In another exemplary embodiment the microvesicles are obtained from mesenchymal stem cells derived from bone marrow.

In a further exemplary embodiment, the microvesicles are obtained from stem cells that are allogenic in origin. In some embodiments, the microvesicles are obtained from stem cells from a donor that is a partial HLA match. In other embodiments, the microvesicles are obtained from stem cells from a donor that is a complete HLA mismatch. In still other embodiments, the microvesicles are obtained from stem cells from a donor that is a match.

In one embodiment, the cells are cells derived from bone-marrow aspirate. In one embodiment, the cells derived from bone marrow aspirate are bone marrow-derived mesenchymal stem cells. In one embodiment, the cells derived from bone marrow aspirate are mononuclear cells. In one embodiment, the cells derived from bone marrow aspirate are a mixture of mononuclear cells and bone marrow-derived mesenchymal stem cells.

In one embodiment, bone marrow-derived mesenchymal stem cells are isolated from bone marrow aspirate by culturing bone marrow aspirate in plastic tissue culture flasks for a period of time of up to about 4 days, followed by a wash to remove the non-adherent cells.

In one embodiment, mononuclear cells are isolated from bone marrow aspirate by low-density centrifugation using a ficoll gradient, and collecting the mononuclear cells at the interface.

In one embodiment, prior to isolation of microvesicles according to the methods of the present disclosure, the cells are cultured, grown or maintained at an appropriate temperature and gas mixture (typically, 37° C., 5% CO₂ for mammalian cells) in a cell incubator. Culture conditions vary widely for each cell type, and are readily determined by one of ordinary skill in the art.

In one embodiment, one, or more than one culture condition is varied. In one embodiment, this variation results in a different phenotype.

In one embodiment, where the cells require serum in their culture medium, to begin the microvesicle isolation procedure, the cell culture medium is supplemented with microvesicle-free serum and then added to the cells to be conditioned. The microvesicles are collected from the conditioned cell culture medium. Serum may be depleted by any suitable method, such as, for example, ultracentrifugation, filtration, precipitation, and the like. The choice of medium, serum concentration, and culture conditions are influenced by a variety of factors readily appreciated by one of ordinary skill in the art, including, for example, the cell type being cultured, the desired purity of the microvesicles, the desired phenotype of the cultured cell, and the like. In one embodiment, the cell culture medium that is conditioned for the microvesicle isolation procedure is the same type of cell culture medium that the cells were grown in, prior to the microvesicle isolation procedure.

In one embodiment, to begin the microvesicle isolation procedure, the cell culture medium is removed, and serum-free medium is added to the cells to be conditioned. The microvesicles are then collected from the conditioned serum free medium. The choice of medium, and culture conditions are influenced by a variety of factors readily appreciated by one of ordinary skill in the art, including, for example, the cell type being cultured, the desired purity of the microvesicles, the desired phenotype of the cultured cell, and the like. In one embodiment, the serum-free medium is supplemented with at least one additional factor that promotes or enhances the survival of the cells in the serum free medium. Such factor may, for example, provide trophic support to the cells, inhibit, or prevent apoptosis of the cells.

The cells are cultured in the culture medium for a period of time sufficient to allow the cells to secrete microvesicles into the culture medium. The period of time sufficient to allow the cells to secrete microvesicles into the culture medium is influenced by a variety of factors readily appreciated by one of ordinary skill in the art, including, for example, the cell type being cultured, the desired purity of the microvesicles, the desired phenotype of the cultured cell, desired yield of microvesicles, and the like.

The microvesicles are then removed from the culture medium by the methods of the present disclosure.

In one embodiment, prior to the microvesicle isolation procedure, the cells are treated with at least one agent selected from the group consisting of an anti-inflammatory compound, an anti-apoptotic compound, an inhibitor of fibrosis, a compound that is capable of enhancing angiogenesis, an immunosuppressive compound, a compound that promotes survival of the cells, a chemotherapeutic, a compound capable of enhancing cellular migration, a neurogenic compound, and a growth factor. In one embodiment, while the cells are being cultured in the medium from which the microvesicles are collected, the cells are treated with at least one agent selected from the group consisting of an anti-inflammatory compound, an anti-apoptotic compound, an inhibitor of fibrosis, a compound that is capable of enhancing angiogenesis, an immunosuppressive compound, a compound that promotes survival of the cells, and a growth factor.

In one embodiment, the anti-inflammatory compound may be selected from the compounds disclosed in U.S. Pat. No. 6,509,369.

In one embodiment, the anti-apoptotic compound may be selected from the compounds disclosed in U.S. Pat. No. 6,793,945.

In one embodiment, the inhibitor of fibrosis may be selected from the compounds disclosed in U.S. Pat. No. 6,331,298.

In one embodiment, the compound that is capable of enhancing angiogenesis may be selected from the compounds disclosed in U. S. Patent Application 2004/0220393 or U. S. Patent Application 2004/0209901.

In one embodiment, the immunosuppressive compound may be selected from the compounds disclosed in U. S. Patent Application 2004/0171623.

In one embodiment, the compound that promotes survival of the cells may be selected from the compounds disclosed in U. S. Patent Application 2010/0104542.

In one embodiment, the growth factor may be at least one molecule selected from the group consisting of members of the TGF-β family, including TGF-β1, 2, and 3, bone morphogenic proteins (BMP-2, -3, -4, -5, -6, -7, -11, -12, and -13), fibroblast growth factors-1 and -2, platelet-derived growth factor-AA, -AB, and -BB, platelet rich plasma, insulin growth factor (IGF-I, II) growth differentiation factor (GDF-5, -6, -8, -10, -15), vascular endothelial cell-derived growth factor (VEGF), pleiotrophin, endothelin, among others. Other pharmaceutical compounds can include, for example, nicotinamide, hypoxia inducible factor 1-alpha, glucagon like peptide-1 (GLP-1), GLP-1 and GLP-2 mimetibody, and II, Exendin-4, nodal, noggin, NGF, retinoic acid, parathyroid hormone, tenascin-C, tropoelastin, thrombin-derived peptides, cathelicidins, defensins, laminin, biological peptides containing cell- and heparin-binding domains of adhesive extracellular matrix proteins such as fibronectin and vitronectin, and MAPK inhibitors, such as, for example, compounds disclosed in U. S. Patent Application 2004/0209901 and U. S. Patent Application 2004/0132729.

In one embodiment, microvesicles are isolated from a biological fluid comprising cell culture medium conditioned using a culture of bone marrow-derived mesenchymal stem cells comprising the steps of:
a) obtaining a population of bone marrow-derived mesenchymal stem cells and seeding flasks at a 1:4 dilution of cells,
b) culturing the cells in medium until the cells are 80 to 90% confluent,
c) removing and clarifying the medium to remove cellular debris,
d) precipitating the microvesicles by adding a precipitating agent to the clarified culture medium,
e) collecting the precipitated microvesicles and washing the material to remove the precipitating agent, and
f) suspending the washed microvesicles in a solution for storage or subsequent use.

In one embodiment, microvesicles are isolated from a biological fluid comprising cell culture medium conditioned using a culture of bone marrow-derived mononuclear cells comprising the steps of:
a) obtaining a population of bone marrow-derived mononuclear cells and seeding flasks at a 1:4 dilution of cells,
b) culturing the cells in medium until the cells are 80 to 90% confluent,
c) removing and clarifying the medium to remove cellular debris,
d) precipitating the microvesicles by adding a precipitating agent to the clarified culture medium,
e) collecting the precipitated microvesicles and washing the material to remove the precipitating agent, and
f) suspending the washed microvesicles in a solution for storage or subsequent use.

In one embodiment, the bone marrow-derived mesenchymal stem cells are cultured in medium comprising α-MEM supplemented with 20% fetal bovine serum and 1% penicillin/streptomycin/glutamine at 37° C. in 95% humidified air and 5% CO₂.

In one embodiment, the bone marrow-derived mononuclear cells are cultured in medium comprising α-MEM supplemented with 20% fetal bovine serum and 1% penicillin/streptomycin/glutamine at 37° C. in 95% humidified air and 5% CO₂.

In one embodiment, the medium is clarified by centrifugation.

In one embodiment, the precipitating agent is polyethylene glycol having an average molecular weight of 6000. In one embodiment, the polyethylene glycol is used at a concentration of about 8.5 w/v %. In one embodiment, the polyethylene glycol is diluted in a sodium chloride solution having a final concentration of 0.4 M.

In one embodiment, the precipitated microvesicles are collected by centrifugation.

In one embodiment, the isolated microvesicles are washed via centrifugal filtration, using a membrane with a 100 kDa molecular weight cut-off, using phosphate buffered saline.

Biological fluid comprising plasma: In one embodiment, microvesicles are obtained from plasma. The plasma may be obtained from a healthy individual, or, alternatively, from an individual with a particular disease phenotype.

In one embodiment, microvesicles are isolated from a biological fluid comprising plasma comprising the steps of:
a) obtaining plasma and diluting the plasma with cell culture medium,
b) precipitating the microvesicles by adding a precipitating agent to the diluted plasma,
c) collecting the precipitated microvesicles and washing the material to remove the precipitating agent, and
d) suspending the washed microvesicles in a solution for storage or subsequent use.

In one embodiment, the plasma is diluted 1:10 with culture medium. In one embodiment, the culture medium is α-MEM.

In one embodiment, the precipitating agent is polyethylene glycol having an average molecular weight of 6000. In one embodiment, the polyethylene glycol is used at a concentration of about 8.5 w/v %. In one embodiment, the polyethylene glycol is diluted in a sodium chloride solution having a final concentration of 0.4 M.

In one embodiment, the precipitated microvesicles are collected by centrifugation.

In one embodiment, the isolated microvesicles are washed via centrifugal filtration, using a membrane with a 100 kDa molecular weight cut-off, using phosphate buffered saline.

Biological fluid comprising bone marrow aspirate: In one embodiment, microvesicles are obtained from bone marrow aspirate. In one embodiment, microvesicles are obtained from the cellular fraction of the bone marrow aspirate. In one embodiment, microvesicles are obtained from the acellular fraction of the bone marrow aspirate.

In one embodiment, microvesicles are obtained from cells cultured from bone marrow aspirate. In one embodiment, the cells cultured from bone marrow aspirate are used to condition cell culture medium, from which the microvesicles are isolated.

In one embodiment, microvesicles are isolated from a biological fluid comprising bone marrow aspirate comprising the steps of:
a) obtaining bone marrow aspirate and separating the bone marrow aspirate into an acellular portion and a cellular portion,
b) diluting the acellular portion,
c) clarifying the diluted acellular portion to remove cellular debris,
d) precipitating the microvesicles in the acellular portion by adding a precipitating agent to the diluted acellular portion,
e) collecting the precipitated microvesicles and washing the material to remove the precipitating agent, and
f) suspending the washed microvesicles in a solution for storage or subsequent use.

In one embodiment, the acellular portion is diluted 1:10 with culture medium.

In one embodiment, the culture medium is □-MEM.

In one embodiment, the diluted acellular portion is clarified by centrifugation.

In one embodiment, the precipitating agent is polyethylene glycol having an average molecular weight of 6000. In one embodiment, the polyethylene glycol is used at a concentration of about 8.5 w/v %. In one embodiment, the polyethylene glycol is diluted in a sodium chloride solution having a final concentration of 0.4 M.

In one embodiment, the precipitated microvesicles are collected by centrifugation.

In one embodiment, the isolated microvesicles are washed via centrifugal filtration, using a membrane with a 100 kDa molecular weight cut-off, using phosphate buffered saline.

In one embodiment the cellular portion is further processed to isolate and collect cells. In one embodiment, the cellular portion is further processed to isolate and collect bone marrow-derived mesenchymal stem cells. In one embodiment, the cellular portion is further processed to isolate and collect bone marrow-derived mononuclear cells. In one embodiment, the cellular portion is used to condition medium, from which microvesicles may later be derived.

In one embodiment, microvesicles are isolated from the cellular portion. The cellular portion may be incubated for a period of time prior to the isolation of the microvesicles. Alternatively, the microvesicles may be isolated from the cellular portion immediately after the cellular portion is collected.

In one embodiment, the cellular portion is also treated with at least one agent selected from the group consisting of an anti-inflammatory compound, an anti-apoptotic compound, an inhibitor of fibrosis, a compound that is capable of enhancing angiogenesis, an immunosuppressive compound, a compound that promotes survival of the cells, a chemotherapeutic, a compound capable of enhancing cellular migration, a neurogenic compound, and a growth factor.

In one embodiment, the anti-inflammatory compound may be selected from the compounds disclosed in U.S. Pat. No. 6,509,369.

In one embodiment, the anti-apoptotic compound may be selected from the compounds disclosed in U.S. Pat. No. 6,793,945.

In one embodiment, the inhibitor of fibrosis may be selected from the compounds disclosed in U.S. Pat. No. 6,331,298.

In one embodiment, the compound that is capable of enhancing angiogenesis may be selected from the compounds disclosed in U. S. Patent Application 2004/0220393 or U. S. Patent Application 2004/0209901.

In one embodiment, the immunosuppressive compound may be selected from the compounds disclosed in U. S. Patent Application 2004/0171623.

In one embodiment, the compound that promotes survival of the cells may be selected from the compounds disclosed in U. S. Patent Application 2010/0104542.

In one embodiment, the growth factor may be at least one molecule selected from the group consisting of members of the TGF-β family, including TGF-β1, 2, and 3, bone morphogenic proteins (BMP-2, -3, -4, -5, -6, -7, -11, -12, and -13), fibroblast growth factors-1 and -2, platelet-derived growth factor-AA, -AB, and -BB, platelet rich plasma, insulin growth factor (IGF-I, II) growth differentiation factor (GDF-5, -6, -8, -10, -15), vascular endothelial cell-derived growth factor (VEGF), pleiotrophin, endothelin, among others. Other pharmaceutical compounds can include, for example, nicotinamide, hypoxia inducible factor 1-alpha, glucagon like peptide-1 (GLP-1), GLP-1 and GLP-2 mimetibody, and II, Exendin-4, nodal, noggin, NGF, retinoic acid, parathyroid hormone, tenascin-C, tropoelastin, thrombin-derived peptides, cathelicidins, defensins, laminin, biological peptides containing cell- and heparin-binding domains of adhesive extracellular matrix proteins such as fibronectin and vitronectin, and MAPK inhibitors, such as, for example, compounds disclosed in U. S. Patent Application 2004/0209901 and U. S. Patent Application 2004/0132729. In one embodiment, the cellular portion is cultured under hypoxic conditions. In one embodiment, the cellular portion is heat-shocked.

Biological fluid comprising urine: In one embodiment, microvesicles are obtained from urine. The urine may be obtained from a healthy individual, or, alternatively, from an individual with a particular disease phenotype.

In one embodiment, microvesicles are isolated from a biological fluid comprising urine comprising the steps of:
a) obtaining a urine sample,
b) clarifying the urine to remove cellular debris,
c) precipitating the microvesicles by adding a precipitating agent to the clarified urine,
d) collecting the precipitated microvesicles and washing the material to remove the precipitating agent, and
e) suspending the washed microvesicles in a solution for storage or subsequent use.

In one embodiment, the urine is clarified by centrifugation.

In one embodiment, the precipitating agent is polyethylene glycol having an average molecular weight of 6000. In one embodiment, the polyethylene glycol is used at a concentration of about 8.5 w/v %. In one embodiment, the polyethylene glycol is diluted in a sodium chloride solution having a final concentration of 0.4 M.

In one embodiment, the precipitated microvesicles are collected by centrifugation.

In one embodiment, the isolated microvesicles are washed via centrifugal filtration, using a membrane with a 100 kDa molecular weight cut-off, using phosphate buffered saline.

In an alternate embodiment of the present disclosure, the biological fluids are clarified by filtration. In an alternate embodiment, the precipitated microvesicles are collected by filtration. In an alternate embodiment, the biological fluids are clarified and the precipitated microvesicles are collected by filtration. In certain embodiments, filtration of either the biological fluid, and/or the precipitated microvesicles required the application of an external force. The external force may be gravity, either normal gravity or centrifugal force. Alternatively, the external force may be suction.

In one embodiment, the present embodiment provides an apparatus to facilitate the clarification of the biological fluid by filtration. In one embodiment, the present disclosure provides an apparatus to facilitate collection of the precipitated microvesicles by filtration. In one embodiment, the present disclosure provides an apparatus that facilitates the clarification of the biological fluid and the collection of the precipitated microvesicles by filtration. In one embodiment, the apparatus also washes the microvesicles.

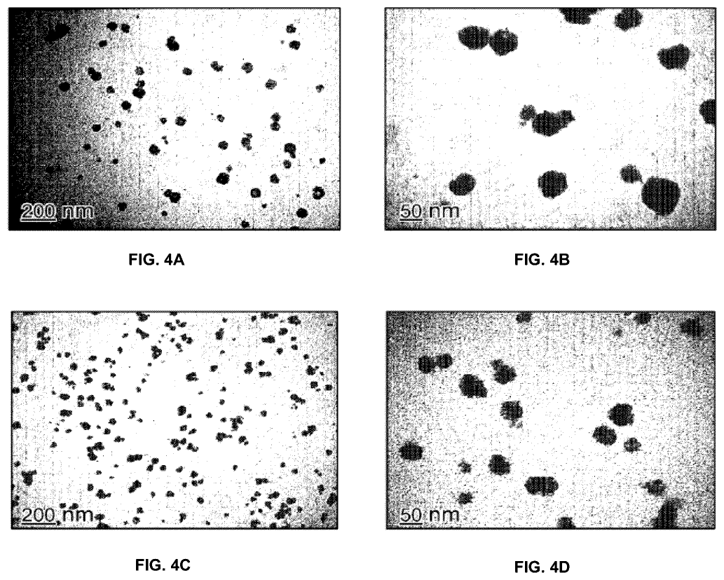

In one embodiment, the apparatus is the apparatus shown in FIG. 4. In this embodiment, the biological fluid is added to the inner chamber. The inner chamber has a first filter with a pore size that enables the microvesicles to pass, while retaining any particle with a size greater than a microvesicle in the inner chamber. In one embodiment, the pore size of the filter of the inner chamber is 1 μm. In this embodiment, when the biological fluid passed from the inner chamber through the filter, particles greater than 1 μm are retained in the inner chamber, and all other particles collect in the region between the bottom of the inner chamber and a second filter.

The second filter has a pore size that does not allow microvesicles to pass. In one embodiment, the pore size of the second filter of the inner chamber is 0.01 μm. In this embodiment, when the biological fluid passed through the second filter, the microvesicles are retained in the region between the bottom of the inner chamber and the second filter, and all remaining particles and fluid collect in the bottom of the apparatus.

One of ordinary skill in the art can readily appreciate that the apparatus can have more than two filters, of varying pore sizes to select for microvesicles of desired sizes, for example.

In one embodiment, a precipitating agent is added to the biological fluid in the inner chamber. In one embodiment, a precipitating agent is added to the filtrate after it has passed through the first filter. The filter membranes utilized by the apparatus of the present disclosure may be made from any suitable material, provided the filter membrane does not react with the biological fluid, or bind with components within the biological fluid. For example, the filter membranes may be made from a low bind material, such as, for example, polyethersulfone, nylon6, polytetrafluoroethylene, polypropylene, zeta modified glass microfiber, cellulose nitrate, cellulose acetate, polyvinylidene fluoride, regenerated cellulose.

### The Microvesicles of the Present Disclosure

In one embodiment, the microvesicles of the present disclosure have a size of about 2 nm to about 5000 nm. In an alternate embodiment, the microvesicles of the present disclosure have a size of about 2 nm to about 1000 nm. In an alternate embodiment, the microvesicles of the present disclosure have a size of about 2 nm to about 500 nm. In an alternate embodiment, the microvesicles of the present disclosure have a size of about 2 nm to about 400 nm. In an alternate embodiment, the microvesicles of the present disclosure have a size of about 2 nm to about 300 nm. In an alternate embodiment, the microvesicles of the present disclosure have a size of about 2 nm to about 200 nm. In an alternate embodiment, the microvesicles of the present disclosure have a size of about 2 nm to about 100 nm. In an exemplary embodiment, the isolated microvesicles range in size from about 100 nm to about 800 nm. In another exemplary embodiment, the isolated microvesicles range in size from about 150 nm to about 700 nm. In further exemplary embodiment, the isolated microvesicles range in size from about 200 nm to about 700 nm. The size of the microvesicles can be determined by for example, electron microscopy and nanoparticle tracking analysis such as NanoSight™.

In one embodiment, the microvesicles of the present disclosure have a molecular weight of at least 100 kDa.

Microvesicles isolated according to the methods of the present disclosure may be used for therapies. Alternatively, microvesicles isolated according to the methods of the present disclosure may be used for diagnostic tests. Alternatively, the microvesicles of the present disclosure may be used to alter or engineer cells or tissues. In the case where the microvesicles of the present disclosure are used to alter or engineer cells or tissues, the microvesicles may be loaded, labeled with RNA, DNA, lipids, carbohydrates, protein, drugs, small molecules, metabolites, subcellular organelles or combinations thereof, that will alter or engineer a cell or tissue. Alternatively, the microvesicles may be isolated from cells or tissues that express and/or contain the RNA, DNA, lipids, carbohydrates, protein, drugs, small molecules, metabolites, subcellular organelles or combinations thereof.

### Use of the Microvesicles of the Present Disclosure in Therapies

The microvesicles of the present disclosure can be used to prevent or treat GVHD or related conditions.

In some embodiments, the GVHD is acute GVHD. In other embodiments the GVHD is chronic GVHD.

In still other embodiments, the GVHD is refractory to another GVHD treatment. In exemplary embodiments, the GVHD is refractory to an immune modulator or an immune suppressive drug. In some embodiments, the GVHD is refractory to a treatment selected from the group consisting of steroids (e.g., corticosteroids), anti-metabolites (e.g., methotrexate), calcineurin inhibitors (e.g., tacrolimus and cyclosporine), mTOR inhibitors (e.g., rapamycin), kinase inhibitors (e.g., Janus kinase), signal transducer and activator of transcription (STAT) inhibitors (e.g., ruxolitinib (RUX), nucleotide analog inhibitors (e.g., mycophenolate mofetil), monoclonal and/or polyclonal antibody preparations (e.g., antithymocyte globulins including thymoglobulin).

In further embodiments, the GVHD is the result of a subject receiving a bone marrow transplant or stem cell transplant.

In one embodiment, the microvesicles of the present disclosure are used to promote or enhance wound healing. In one embodiment, the wound is a cutaneous wound. In other embodiment, the wound is a subcutaneous wound.

In one embodiment, the microvesicles of the present disclosure are used to reduce inflammation. In one embodiment, the inflammation is cutaneous. In other embodiment, inflammation is subcutaneous.

In some embodiments, the presently disclosed microvesicles increase the number of regulatory t cells (Tregs) in the tissue of a subject. In an exemplary embodiment, the presently disclosed microvesicles increase the number of regulatory t cells (Tregs) in the skin tissue of a subject. In a further exemplary embodiment, the Tregs are FOXP3+.

In one embodiment, the microvesicles of the present disclosure are used to orchestrate complex tissue regeneration in a patient. In some embodiments, the tissue is cutaneous. In other embodiments, the tissue is subcutaneous.

In one embodiment, the present disclosure provides an isolated preparation of microvesicles that can promote functional regeneration and organization of complex tissue structures. In one embodiment the present disclosure provides an isolated preparation of microvesicles that can regenerate at least one tissue in a patient with diseased, damages or missing skin selected from the group consisting of: epithelial tissue, stromal tissue, nerve tissue, vascular tissue and adnexal structures. In one embodiment, the present disclosure provides an isolated preparation of microvesicles that can regenerate tissue and/or cells from all three germ layers. In an exemplary embodiment, the present disclosure provides an isolated preparation of microvesicles that promotes the restoration of immune regulatory organs, such as the thymus, spleen, gut skin and bone marrow. In a further exemplary embodiment, in an individual with immune regulatory organs that are damaged (i.e., from chemotherapy, radiation, or other treatment), the present disclosure provides an isolated preparation of microvesicles that promotes the restoration of the damaged immune regulatory organs.

In one embodiment, the present disclosure provides an isolated preparation of microvesicles that is used to modulate the immune system of a patient.

In one embodiment, the present disclosure provides an isolated preparation of microvesicles that is used to treat weight loss caused by GVHD in a subject.

In one embodiment, the present disclosure provides an isolated preparation of microvesicles that enhances the survival of tissue or cells that is transplanted into a patient. In one embodiment, the patient is treated with the isolated preparation of microvesicles prior to receiving the transplanted tissue or cells. In an alternate embodiment, the patient is treated with the isolated preparation of microvesicles after receiving the transplanted tissue or cells. In an alternate embodiment, the tissue or cells is treated with the isolated preparation of microvesicles. In one embodiment, the tissue or cells is treated with the isolated preparation of microvesicles prior to transplantation.

In one embodiment, the microvesicles of the present disclosure are used to deliver molecules to cells. The delivery of molecules may be useful in treating or preventing a disease, i.e., GVHD. In one embodiment, the delivery is according to the methods described in PCT Application WO04014954A1. In an alternate embodiment, the delivery is according to the methods described in PCT Application WO2007126386A1. In an alternate embodiment, the delivery is according to the methods described in PCT Application WO2009115561A1. In an alternate embodiment, the delivery is according to the methods described in PCT Application WO2010119256A1.

In one embodiment, the present disclosure provides an isolated preparation of microvesicles containing at least one molecule selected from the group consisting of RNA, DNA, lipid, carbohydrate, metabolite, protein, and combination thereof from a host cell. In one embodiment, the host cell is engineered to express at least one molecule selected from the group consisting of RNA, DNA, lipid, carbohydrate, metabolite, protein, and combination thereof. In one embodiment, the isolated preparation of microvesicles containing at least one molecule selected from the group consisting of RNA, DNA, lipid, carbohydrate, metabolite, protein, and combination thereof from a host cell is used as a therapeutic agent.

In one embodiment, the microvesicles of the present disclosure are administered in combination with an additional therapeutic agent for treating GVHD. In some embodiments, the additional therapeutic is an immunosuppressant. In some embodiments, the additional therapeutic agent is selected from the group consisting of steroids (e.g., corticosteroids), anti-metabolites (e.g., methotrexate), calcineurin inhibitors (e.g., tacrolimus and cyclosporine), mTOR inhibitors (e.g. rapamycin), kinase inhibitors (e.g., Janus kinase), signal transducer and activator of transcription (STAT) inhibitors (e.g., ruxolitinib (RUX)), and nucleotide analog inhibitors (e.g., mycophenolate mofetil.) In additional embodiments, treatment of the subject with the isolated microvesicles lowers the dose of the additional treatment required to treat GVHD in the subject. In certain exemplary embodiments, the additional therapeutic agent is a systemic corticosteroid. In some embodiments, the additional therapeutic agent is a systemic corticosteroid that is administered daily.

### Administration Regimens

According to certain embodiments, multiple doses of the pharmaceutical composition comprising isolated microvesicles may be administered to a subject over a defined time course. Such methods comprise sequentially administering to a subject multiple doses of the isolated microvesicles. As used herein, “sequentially administering” means that each dose of the isolated microvesicles are administered to the subject at a different point in time, e.g., on different days separated by a predetermined interval (e.g., hours, days, weeks, or months). Methods that comprise sequentially administering to the patient a single initial dose of isolated microvesicles, followed by one or more secondary doses of the isolated microvesicles, and optionally followed by one or more tertiary doses of the isolated microvesicles, are provided.

In some embodiments, the isolated microvesicles are administered to a subject 1, 2, 3, 4, 5, 6, 7, 8, 9, 10, 11, 12, 13, or 14 times per week. In exemplary embodiments, the isolated microvesicles are administered to a subject once per day.

In other embodiments, the isolated microvesicles are administered to a subject 1, 2, 3, 4, 5, 6, 7, 8, 9, 10, 11, 12, 13, 14, or 15 times per month.

In some embodiments, the isolated microvesicles are administered to a subject for a period of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 76, 77, 78, 79, 80, 81, 82, 83, 84, 85, 86, 87, 88, 89, 90, 91, 92, 93, 94, 95, 96, 97, 98, 99, or 100 days. In other embodiments, the isolated microvesicles are administered to a subject for a period of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks. In other embodiments, the isolated microvesicles are administered to a subject for a period of years, such as about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more years. In an exemplary embodiment, the isolated microvesicles are administered to a subject for about 50 days.

In some embodiments, the isolated microvesicles are administered to a subject as a prophylactic treatment and the isolated microvesicles are administered to the subject before the subject experiences symptoms of GVHD (i.e., before the subject receives a transplant.) In various embodiments, the isolated microvesicles are administered to a subject as a prophylactic treatment in a time frame between the day the subject receives a transplant and about 100 days after the subject receives a transplant.

In some embodiments, the isolated microvesicles are administered to a subject as a treatment for GVHD and the isolated microvesicles are administered to the subject after the subject experiences symptoms of GVHD (i.e., after the subject receives a transplant.) In various embodiments, the isolated microvesicles are administered to a subject as a GVHD treatment in a time frame between the day the subject receives the transplant and about 1.5 years after the subject receives the transplant.

In certain embodiments, the isolated microvesicles are administered to a subject in dose provided in mg/kg, wherein the mass of isolated microvesicles administered to the subject is determined by the subject's body weight. In some embodiments, the isolated microvesicles are administered in a dose between about 0.01 mg/kg to 100 mg/kg. In some embodiments the isolated microvesicles are administered in a dose of about 0.01, about 0.1, about 1, about 10, or about 100 mg/kg.

In certain embodiments the isolated microvesicles are administered to a subject in a dose provided in mg per mm² of body surface area, wherein the mass of isolated microvesicles administered to the subject is determined by the surface area of the skin onto which the isolated microvesicles are administered.

In some embodiments, the dosage of isolated microvesicles administered to a subject is determined by whether the isolated microvesicles are administered as a prophylactic treatment or after the onset of GVHD symptoms.

### Use of the Microvesicles of the Present Disclosure in Pharmaceutical Compositions

For therapeutic use, isolated microvesicles are preferably combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Accordingly, microvesicle compositions of the present disclosure can comprise at least one of any suitable excipients, such as, but not limited to, diluent, binder, stabilizer, buffers, salts, lipophilic solvents, preservative, adjuvant or the like. Pharmaceutically acceptable excipients are preferred. Non-limiting examples of, and methods of preparing such sterile solutions are well known in the art, such as, but not limited to, those described in Gennaro, Ed., Remington's Pharmaceutical Sciences, 18th Edition, Mack Publishing Co. (Easton, Pa.) 1990. Pharmaceutically acceptable carriers can be routinely selected that are suitable for the mode of administration, solubility and/or stability of microvesicle composition as well known in the art or as described herein.

Pharmaceutical excipients and additives useful in the present composition include but are not limited to proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody molecule components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like.

Carbohydrate excipients suitable for use in the disclosure include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present disclosure are mannitol, trehalose, and raffinose.

Microvesicle compositions can also include a buffer or a pH adjusting agent; typically, the buffer is a salt prepared from an organic acid or base. Representative buffers include organic acid salts such as salts of citric acid, acetic acid, ascorbic acid, gluconic acid, carbonic acid, tartaric acid, succinic acid, or phthalic acid; Tris, tromethamine hydrochloride, or phosphate buffers.

Additionally, microvesicle compositions of the disclosure can include polymeric excipients/additives such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the antibody molecule compositions according to the disclosure are known in the art, e.g., as listed in “Remington: The Science & Practice of Pharmacy,” 19th ed., Williams & Williams, (1995), and in the “Physician's Desk Reference,” 52nd ed., Medical Economics, Montvale, N.J. (1998). Preferred carrier or excipient materials are carbohydrates (e.g., saccharides and alditols) and buffers (e.g., citrate) or polymeric agents.

The present disclosure provides for stable compositions, comprising isolated microvesicles in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3, 0.4, 0.5, 0.9, or 1.0%), 0.1-3% benzyl alcohol (e.g., 0.5, 0.9, 1.1, 1.5, 1.9, 2.0, or 2.5%), 0.001-0.5% thimerosal (e.g., 0.005 or 0.01%), 0.001-2.0% phenol (e.g., 0.05, 0.25, 0.28, 0.5, 0.9, or 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, or 1.0%), and the like.

Pharmaceutical compositions containing isolated microvesicles as disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. A preferred route of administration for isolated microvesicles is topical administration. Useful formulations can be prepared by methods known in the pharmaceutical art. For example, see Remington's Pharmaceutical Sciences (1990) supra. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations are preferably sterile. Sterilization can be accomplished by any suitable method, e.g., filtration through sterile filtration membranes.

The compositions of this disclosure may be in a variety of forms. These include, for example, liquid, semi-solid and solid dosage forms, such as liquid solutions (e.g., injectable and infusible solutions), dispersions or suspensions, and liposomes. The preferred form depends on the intended mode of administration and therapeutic application. Typical preferred compositions are in the form of injectable or infusible solutions. In some embodiments, the administration is parenteral (e.g., intravenous, subcutaneous, intraocular, intraperitoneal, intramuscular). The phrases “parenteral administration” and “administered parenterally” as used herein means modes of administration other than enteral and topical administration, usually by injection, and includes, without limitation, intravenous, intramuscular, subcutaneous, intraarterial, intrathecal, intracapsular, intraorbital, intravitreous, intracardiac, intradermal, intraperitoneal, transtracheal, inhaled, subcutaneous, subcuticular, intraarticular, subcapsular, subarachnoid, intraspinal, epidural and intrasternal injection and infusion.

Various delivery systems can be used to administer isolated microvesicles to a subject. In certain exemplary embodiments, administration of isolated microvesicles is topical. In some embodiments, the isolated microvesicles are administered topically using a dressing, bandage, medical tape, pad, gauze, or similar delivery device.

In other embodiments, the isolated microvesicles are administered by pulmonary delivery, e.g., by intranasal administration, or by oral inhalative administration. Pulmonary delivery may be achieved via a syringe or an inhaler device (e.g., a nebulizer, a pressurized metered-dose inhaler, a multi-dose liquid inhaler, a thermal vaporization aerosol device, a dry powder inhaler or the like). Suitable methods for pulmonary delivery are well-known in the art and are commercially available.

In an exemplary embodiment, the isolated microvesicles are administered by systemic administration. In some embodiments, the isolated microvesicles are administered by intravenous administration.

In another exemplary embodiment, the isolated microvesicles are administered by local injection. In another embodiment, the isolated microvesicles are administered by intramuscular or subcutaneous injection.

In other embodiments, the isolated microvesicles are administered by ocular administration. In an exemplary embodiment, the isolated microvesicles are administered within an eye drop.

In still other embodiments, the isolated microvesicles are administered by oral administration. In an exemplary embodiment, the isolated microvesicles are administered within a pill or tablet.

The present disclosure provides a kit, comprising packaging material and at least one vial comprising a solution of isolated microvesicles with the prescribed buffers and/or preservatives, optionally in an aqueous diluent. The aqueous diluent optionally further comprises a pharmaceutically acceptable preservative. Preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g. isotonicity agents, buffers, antioxidants, preservative enhancers, can be optionally and preferably added to the diluent. An isotonicity agent, such as glycerin, is commonly used at known concentrations. A physiologically tolerated buffer can be added to provide improved pH control. The formulations can cover a wide range of pHs, such as from about pH 4.0 to about pH 10.0, from about pH 5.0 to about pH 9.0, or about pH 6.0 to about pH 8.0.

Other additives, such as a pharmaceutically acceptable solubilizers like TWEEN 20 (polyoxyethylene (20) sorbitan monolaurate), TWEEN 40 (polyoxyethylene (20) sorbitan monopalmitate), TWEEN 80 (polyoxyethylene (20) sorbitan monooleate), Pluronic F68 (polyoxyethylene polyoxypropylene block copolymers), and PEG (polyethylene glycol) or non-ionic surfactants such as polysorbate 20 or 80 or poloxamer 184 or 188, Pluronic® polyls, other block co-polymers, and chelators such as EDTA and EGTA can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

Any of the formulations described above can be stored in a liquid or frozen form and can be optionally subjected to a preservation process.

In certain exemplary embodiments of the disclosure, isolated microvesicles described herein are used to deliver one or more bioactive agents to a target cell. The term “bioactive agent” is intended to include, but is not limited to, proteins (e.g., non-membrane-bound proteins), peptides (e.g., non-membrane-bound peptides), transcription factors, subcellular organelles (e.g., rough or smooth endoplasmic reticulum, golgi bodies, secretory vesicles, endosomes, lysosomes, peroxisomes, nuclear materials and mitochondria), nucleic acids and the like, that are expressed in a cell and/or in a cellular fluid and are added during the purification and/or preparation of EVs described herein, and/or pharmaceutical compounds, proteins (e.g., non-membrane-bound proteins), peptides (e.g., non-membrane-bound peptides), transcription factors, nucleic acids and the like, that isolated microvesicles described herein are exposed to during one or more purification and/or preparation steps described herein. In certain embodiments, a bioactive agent is a collagen VII protein, a collagen VII mRNA, a STAT3 signaling activator (e.g., an interferon, epidermal growth factor, interleukin-5, interleukin-6, a MAP kinase, a c-src non-receptor tyrosine kinase or another molecule that phosphorylates and/or otherwise activates STAT3) and/or a canonical Wnt activator (see, e.g., McBride et al. (2017) Transgenic expression of a canonical Wnt inhibitor, kallistatin, is associated with decreased circulating CD19+B lymphocytes in the peripheral blood. International Journal of Hematology, 1-10. DOI: 10.1007/s12185-017-2205-5, incorporated herein by reference in its entirety). In other embodiments, a bioactive agent is one or more pharmaceutical compounds known in the art.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting. All patents, patent applications and references described herein are incorporated by reference in their entireties for all purposes.

### EXAMPLES

### Example 1: Isolation of Microvesicles from Cell Culture Medium by the Methods of the Present Disclosure

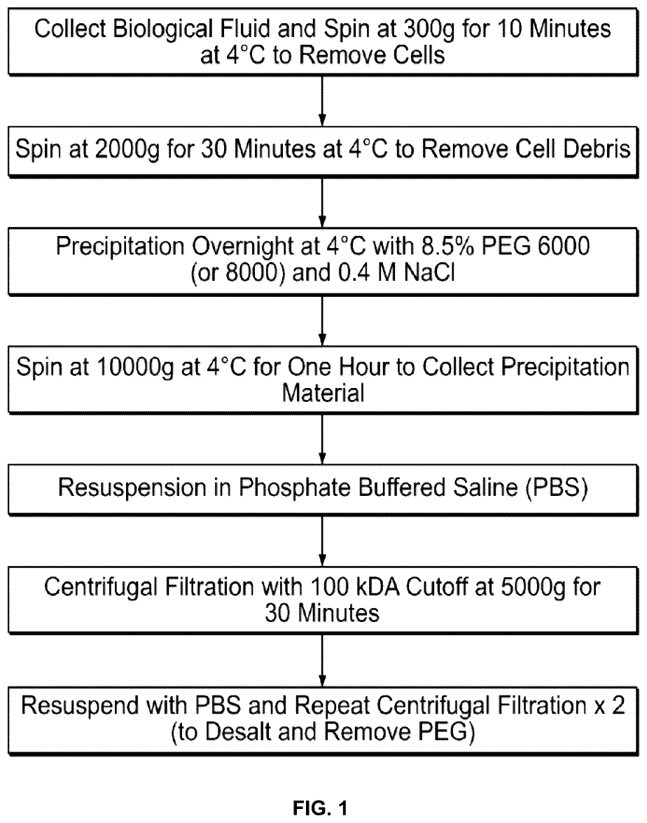

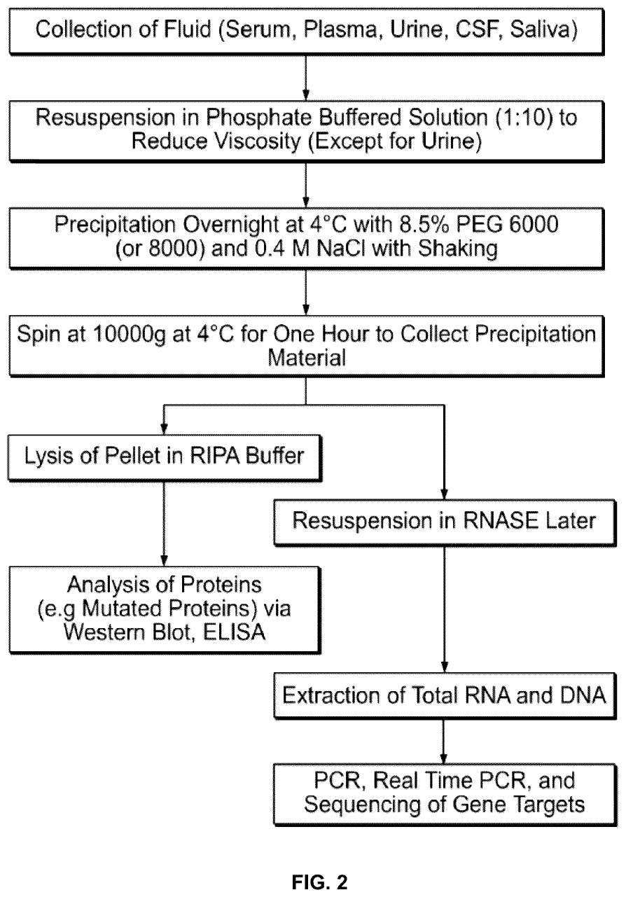

This example illustrates how microvesicles are isolated from cell culture medium by the methods of the present disclosure. An outline of the method to isolate microvesicles from medium that has cultured cells is shown in FIGS. 1 and 2. In summary, the cells are cultured in medium supplemented with microvesicle-free serum (the serum may be depleted of microvesicles by ultracentrifugation, filtration, precipitation, etc.). After culturing the cells for a period of time, the medium is removed and transferred to conical tubes and centrifuged at 400×g for 10 minutes at 4° C. to pellet the cells. Next, the supernatant is transferred to new conical tubes and centrifuged at 2000×g for 30 minutes at 4° C. to further remove cells and cell debris. This may be followed by another centrifugation step (e.g., 10000×g for 30 minutes to further deplete cellular debris and remove larger particles).

Microvesicles are then precipitated at 4° C. using 8.5% w/v PEG 6000 and 0.4 M NaCl. This mixture is spun at 10000×g at 4° C. for 30 minutes. The supernatant is removed and the pellet is resuspended in an appropriate buffer (e.g. PBS). It may be used for immediate downstream reactions or further purified. Further purification procedures can include the use of centrifugal filters (e.g., MWCO of 100 kDa), immunoaffinity, HPLC, tangential flow filtration, phase separation/partitioning, microfluidics, etc.

### Example 2: Isolation of Microvesicles from Culture Medium Conditioned Using Bone Marrow Derived Stem Cells by the Methods of the Present Disclosure

Normal donor human bone marrow was acquired from AllCells LLC (Emeryville, CA, http://www.allcells.com). MSCs were isolated by a standard plastic adherence method. Bone marrow mononuclear cells were isolated by low-density centrifugation using Ficoll-Paque Premium (density: 1.077 g/ml) according to the manufacturer's protocol (GE Healthcare Life Sciences, Pittsburgh, PA). The mononuclear cells were collected at the interface, washed three times in phosphate-buffered saline (PBS) supplemented with 2% FBS (Atlanta Biologics, Atlanta, GA), and resuspended in MSC medium consisting of alpha-minimum essential medium (α-MEM) (Mediatech Inc., Manassas, VA) and 20% FBS, 1% Penicillin/Streptomycin (Lonza, Allendale, NJ) and 1% glutamine (Lonza).

Initial cultures of either MSCs or mononuclear cells were seeded between 2-3×10⁵ cells/cm² in tissue culture-treated dishes (BD Biosciences, San Jose, CA) and placed in a cell incubator at 37° C. in 95% humidified air and 5% CO₂. After 48-72 hours, the non-adherent cells were removed, the culture flasks were rinsed once with PBS, and fresh medium was added to the flask. The cells were grown until 80% confluence was reached and then passaged by Trypsin-EDTA (Life technologies, Carlsbad, CA). Cells were split at a 1:4 ratio into 5-layer multi-flasks (BD Biosciences). Alternatively, cryopreserved MSC were thawed at 37° C. and immediately cultured in α-MEM supplemented with 20% microvesicle-free fetal bovine serum and 1% penicillin/streptomycin/glutamine at 37° C. in 95% humidified air and 5% CO₂. They were expanded similar to above.

The cells were grown in the multi-flasks until 80-90% confluence was reached. The flasks were rinsed twice with PBS and a-MEM supplemented with 1% Penicillin/Streptomycin/Glutamine was added. After 24 hours, the conditioned medium transferred to 50 mL conical centrifuge tubes (Thermo Fisher Scientific Inc., Weston, FL) and immediately centrifuged at 400×g for 10 minutes at 4° C. to pellet any non-adherent cells. The supernatant was transferred to new 50 mL conical centrifuge tubes and centrifuged at 2000×g for 30 minutes at 4° C. to further remove cells and cell debris. The supernatants were collected and placed into 250 ml sterile, polypropylene disposable containers (Corning, Corning, NY). To the supernatant, RNase and protease free polyethylene glycol average molecular weight 6000 (Sigma Aldrich, Saint Louis, MO) at 8.5 w/v % and sodium chloride (final concentration 0.4 M) were added. The solution was placed in a cold room at 4° C. overnight with rocking. The solution was transferred to 50 mL conical centrifuge tubes and centrifuged at 10000×g at 4° C. for 30 minutes. The supernatant was decanted and the microvesicle enriched pellet resuspended in phosphate-buffered saline (PBS). The microvesicle enriched solution was transferred to Amicon ultra-15 centrifugal filter units (nominal molecular weight limit 100 kDa) (Millipore, Billerica, MA) and centrifuged at 5000×g for 30 minutes. The filter units were washed with phosphate-buffered saline and centrifuged again at 5000×g for 30 minutes. The concentrated sample was recovered (approximately 200 μl) from the bottom of the filter device. Protein concentration was determined by the micro BSA Protein assay kit (Pierce, Rockford, IL) and the enriched microvesicle solution was stored at −70 degrees or processed for downstream use (e.g., protein, RNA, and DNA extraction).

### Example 3: Isolation of Microvesicles from Plasma by the Methods of the Present Disclosure

Approximately 6-8 ml of blood (human and pig) was collected via venipuncture and placed into BD Vacutainer plastic EDTA lavender tubes (BD Biosciences, San Jose, CA). The venipuncture tubes were centrifuged at 400×g for 30 minutes at room temperature. Plasma was removed (approximately 3-4 ml) and placed into new 50 ml conical centrifuge tubes (Thermo Fisher Scientific Inc., Weston, FL). Sterile alpha-minimum essential medium (α-MEM) (Mediatech Inc., Manassas, VA) was added in a 1:10 (Plasma to medium) ratio.

To the solution, RNase and protease free polyethylene glycol average molecular weight 6000 (Sigma Aldrich, Saint Louis, MO) at 8.5 w/v % and sodium chloride (final concentration 0.4 M) were added. The solution was placed in a cold room at 4° C. overnight with rocking. The solution was centrifuged at 10000×g at 4° C. for 30 minutes. The supernatant was decanted and the microvesicle enriched pellet resuspended in phosphate-buffered saline (PBS). The microvesicle enriched solution was transferred to Amicon ultra-15 centrifugal filter units (nominal molecular weight limit 100 kDa) (Millipore, Billerica, MA) and centrifuged at 5000×g for 30 minutes. The filter units were washed with phosphate-buffered saline and centrifuged again at 5000×g for 30 minutes. The concentrated sample was recovered (approximately 200-400 □l) from the bottom of the filter device. Protein concentration was determined by the micro BSA Protein assay kit (Pierce, Rockford, IL) and the enriched microvesicle solution was stored at −70 degrees or processed for downstream use (e.g., protein, RNA, and DNA extraction).

### Example 4: Isolation of Microvesicles from Bone Marrow Aspirate by the Methods of the Present Disclosure

Pig bone marrow was isolated from the iliac crest. The skin area was carefully cleaned with povidine iodine 7.5% and isopropanol 70%. An 11-gauge 3 mm trocar (Ranafac, Avon, MA) was inserted into the iliac crest. An aspiration syringe with loaded with 5000-1000 units of heparin to prevent clotting of the marrow sample. Approximately 20-25 ml of marrow was aspirated and the solution transferred to 50 ml conical centrifuge tubes. Alternatively, normal donor human bone marrow (approximately 50 ml) was acquired from AllCells LLC (Emeryville, CA, URL: allcells.com).

The 50 ml conical tubes were centrifuged at 400×g for 30 minutes at room temperature. The supernatant (the acellular portion) was collected (approximately 10-12 ml per 50 ml) and placed into new 50 ml conical centrifuge tubes (Thermo Fisher Scientific Inc., Weston, FL). Sterile alpha-minimum essential medium (α-MEM) (Mediatech Inc., Manassas, VA) was added in a 1:10 (bone marrow supernatant to medium) ratio. The solution was transferred to new 50 ml conical tubes and centrifuged at 2000×g for 30 minutes at 4° C. The supernatant was transferred to new 50 ml conical tubes and to this solution, RNase and protease free polyethylene glycol average molecular weight 6000 (Sigma Aldrich, Saint Louis, MO) at 8.5 w/v % and sodium chloride (final concentration 0.4 M) were added.

The solution was placed in a cold room at 4° C. overnight with rocking. The solution was centrifuged at 10000×g at 4° C. for 30 minutes. The supernatant was decanted and the microvesicle enriched pellet resuspended in phosphate-buffered saline (PBS). The microvesicle enriched solution was transferred to Amicon ultra-15 centrifugal filter units (nominal molecular weight limit 100 kDa) (Millipore, Billerica, MA) and centrifuged at 5000×g for 30 minutes. The filter units were washed with phosphate-buffered saline and centrifuged again at 5000×g for 30 minutes. The concentrated sample was recovered (approximately 200-400 μl) from the bottom of the filter device. Protein concentration was determined by the micro BSA Protein assay kit (Pierce, Rockford, IL) and the enriched microvesicle solution was stored at −70 degrees or processed for downstream use (e.g., protein, RNA, and DNA extraction).

The cellular portion was collected and processed for mesenchymal stem isolation or for bone marrow complete isolation.

### Example 5: Isolation of Microvesicles from Urine by the Methods of the Present Disclosure

Approximately 500 ml of clean catch human urine was isolated and placed into 50 ml conical tubes (Thermo Fisher Scientific Inc., Weston, FL).

The 50 ml conical tubes were centrifuged at 400×g for 30 minutes at 4° C. The supernatant was removed and placed into new 50 ml conical centrifuge tubes (Thermo Fisher Scientific Inc., Weston, FL). The solution was transferred to new 50 ml conical tubes and centrifuged at 2000×g for 30 minutes at 4° C. The supernatant was transferred to new 50 ml conical tubes and to this solution, RNase and protease free polyethylene glycol average molecular weight 6000 (Sigma Aldrich, Saint Louis, MO) at 8.5 w/v % and sodium chloride (final concentration 0.4 M) were added.

The solution was placed in a cold room at 4° C. overnight with rocking. The solution was centrifuged at 10000×g at 4° C. for 30 minutes. The supernatant was decanted and the microvesicle enriched pellet resuspended in phosphate-buffered saline (PBS). The microvesicle enriched solution was transferred to Amicon ultra-15 centrifugal filter units (nominal molecular weight limit 100 kDa) (Millipore, Billerica, MA) and centrifuged at 5000×g for 30 minutes. The filter units were washed with phosphate-buffered saline and centrifuged again at 5000×g for 30 minutes. The concentrated sample was recovered (approximately 200-400 μl) from the bottom of the filter device. Protein concentration was determined by the micro BSA Protein assay kit (Pierce, Rockford, IL) and the enriched microvesicle solution was stored at −70 degrees or processed for downstream use (e.g. protein, RNA, and DNA extraction).

### Example 6: Isolation of Microvesicles from Medium from a Long-Term Culture of Bone Marrow Cells by the Methods of the Present Disclosure

Bone marrow was obtained from an aspirate and red blood cells were lysed using 0.8% ammonium chloride solution containing 0.1 mM EDTA (Stem Cell Technologies, Vancouver, BC). The nucleated cells were pelleted under a fetal bovine serum (Atlanta Biologics, Atlanta, GA) cushion at 400×g for 5 minutes. Nucleated cells were washed in McCoy's 5a media (Mediatech Inc., Manassas, VA) by pelleting at 400×g for 5 min. The cells were resuspended in culture media at a density of 1×10⁶ cells/ml and plated in 25, 75 or 225 cm² flasks (corning, Corning, NY).

Culture media consisted of McCoy's 5a media, 1% sodium bicarbonate (Life technologies, Carlsbad, CA), 0-4% MEM non-essential amino acids (Life technologies), 0-8% MEM essential amino acids (Life technologies), 1% L-glutamine (Lonza, Allendale, NJ), 0.1 μM Hydrocortisone (Life technologies), 1% penicillin/streptomycin (Lonza), 12-5% fetal calf serum (Atlanta Biologics) and 12-5% horse serum (Stem Cell Technology). The cultures were incubated at 33° C. and 5% CO₂. Feeding was performed weekly by adding half of the original volume of media without removing any media during the first nine weeks of culture. If the cultures were grown beyond nine weeks, the volume of culture media was reduced to the original volume and half the original volume of fresh media was added each week.

After approximately nine weeks of culture, the original medium was removed and stored. The cells were washed twice with phosphate buffered saline (PBS) and incubated for 24 hours in media consisting of McCoy's 5a media, 1% sodium bicarbonate, 0-4% MEM nonessential amino acids, 0-8% MEM essential amino acids (Life technologies), 1% L-glutamine (Lonza, Allendale, NJ), and 1% penicillin/streptomycin (Lonza).

After 24 hours, the supernatant was transferred to 50 mL conical centrifuge tubes (Thermo Fisher Scientific Inc., Weston, FL) and immediately centrifuged at 400×g for 10 minutes at 4° C. to pellet any non-adherent cells. The original medium that was stored was added back to the cells. The supernatant were transferred to new 50 mL conical centrifuge tubes and centrifuged at 2000×g for 30 minutes at 4° C. to further remove cells and cell debris.

The supernatant was collected and placed into 250 ml sterile, polypropylene disposable containers (Corning, Corning, NY). To the supernatant, RNase and protease free polyethylene glycol average molecular weight 6000 (Sigma Aldrich, Saint Louis, MO) at 8.5 w/v % and sodium chloride (final concentration 0.4 M) was added. The solution was placed in a cold room at 4° C. overnight with rocking. The solution was transferred to 50 mL conical centrifuge tubes and centrifuged at 10000×g at 4° C. for 30 minutes. The supernatant was decanted and the microvesicle enriched pellet resuspended in phosphate-buffered saline (PBS). The microvesicle enriched solution was transferred to Amicon ultra-15 centrifugal filter units (nominal molecular weight limit 100 kDa) (Millipore, Billerica, MA) and centrifuged at 5000×g for 30 minutes. The filter units were washed with phosphate-buffered saline and centrifuged again at 5000×g for 30 minutes. The concentrated sample was recovered (approximately 200 μl) from the bottom of the filter device. Protein concentration was determined by the micro BSA Protein assay kit (Pierce, Rockford, IL) and the enriched microvesicle solution stored at −70 degrees or processed for downstream use (e.g. protein, RNA, and DNA extraction).

### Example 7: Analysis of the Microvesicles of the Present Disclosure

Samples of microvesicles were analyzed by electron microscopy. For transmission electron microscopy (TEM), each specimen of microvesicles was loaded on formvar-coated, 150 mesh copper grids (Electron Microscopy Sciences, Fort Washington, PA) for 20 minutes. The grids were drained and floated on drops of 2% glutaraldehyde for 5 minutes, then washed in double distilled water (DDOH), followed by staining on drops of 4% aqueous uranyl acetate and multiple washes in DDOH. The grids were examined at 80 kV in a Philips CM10 electron microscope.

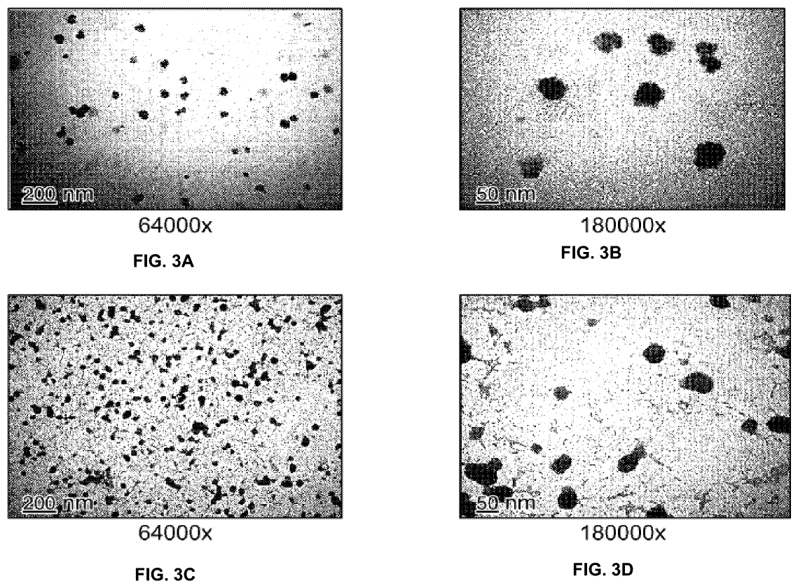

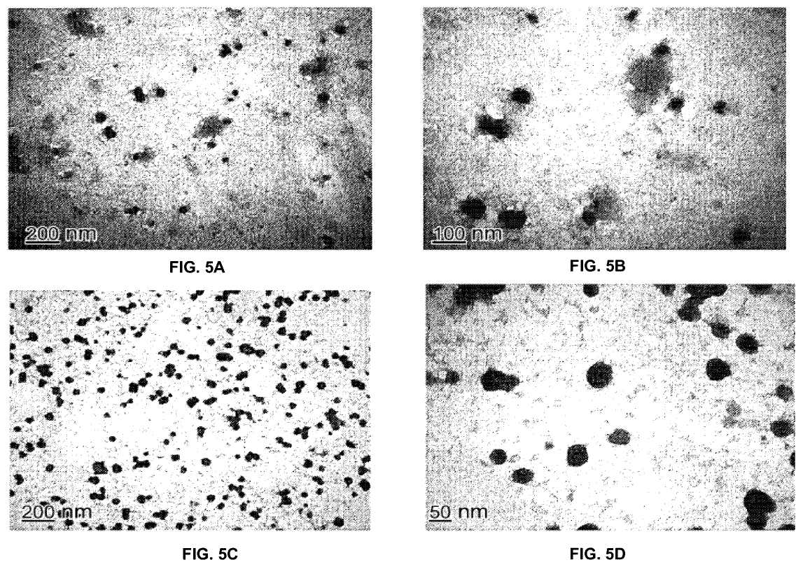

FIG. 3A-C shows electron micrographs of microvesicles derived from human bone marrow-derived mesenchymal stem cells isolated by the ultracentrifuge method (FIGS. 3A and 3B) and according to the methods of the present disclosure as described in Example 1 (FIG. 3C and FIG. 3D). FIG. 4A-C shows electron micrographs of microvesicles derived from porcine bone marrow-derived mesenchymal stem cells isolated by the ultracentrifuge method (FIGS. 4A and 4B) and according to the methods of the present disclosure as described in Example 1 (FIGS. 4C and 4D). FIG. 5A-C shows electron micrographs of microvesicles derived from murine bone marrow-derived mesenchymal stem cells isolated by the ultracentrifuge method (FIGS. 5A and 5B) and according to the methods of the present disclosure as described in Example 1 (FIG. 5C and FIG. 5D).

FIG. 3 to FIG. 5 illustrate the differences between microvesicles isolated by the methods of the present disclosure compared to ultracentrifuge isolation. The microvesicles isolated according to the methods of the present disclosure have borders that are smoother, uncorrugated and appear more “intact.”

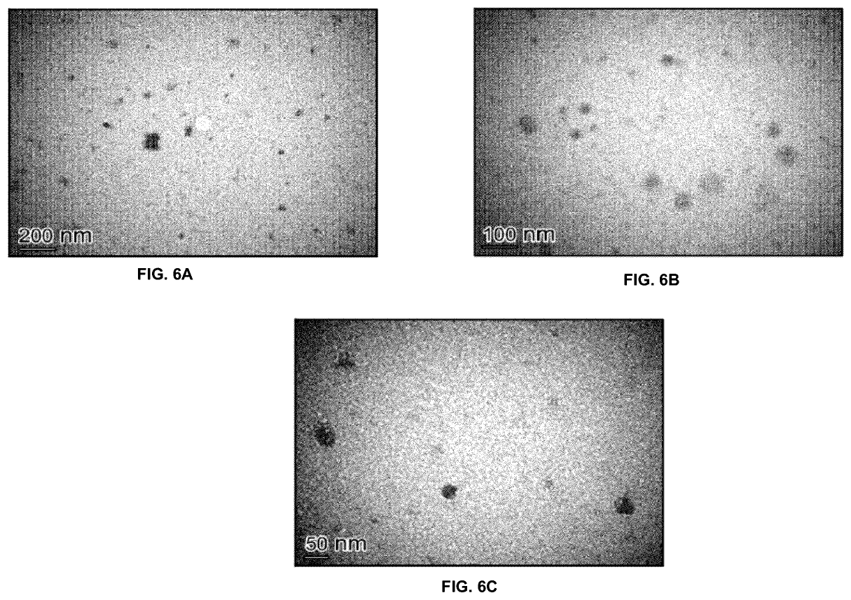

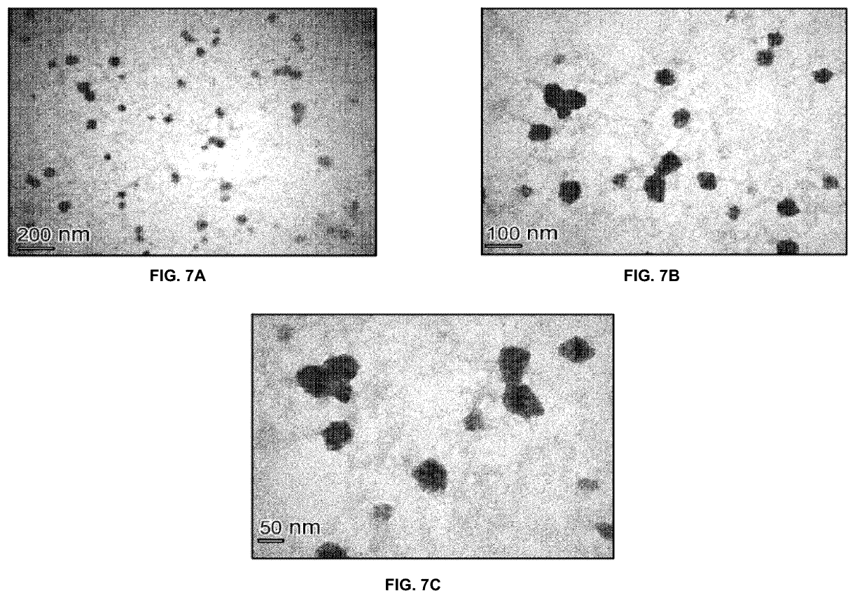

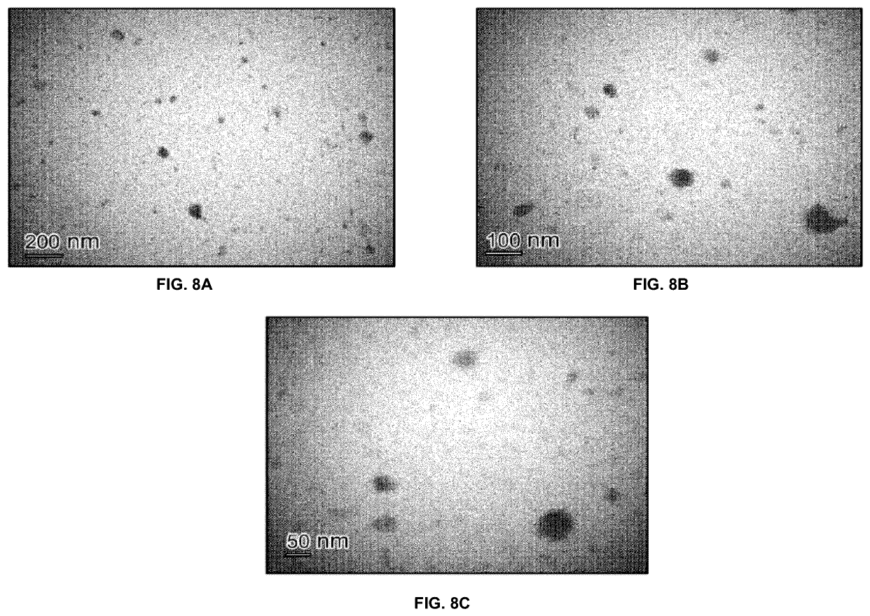

FIG. 6A-C shows electron micrographs of microvesicles isolated from human plasma according to the methods of the present disclosure. The heterogeneity of the shapes and sizes achieved with PEG isolation suggests that all types of microvesicles were isolated. Similar heterogeneity was observed in microvesicles from porcine plasma FIG. 7A-C and human urine FIG. 8A-C that were isolated according to the methods of the present disclosure.

To analyze protein expression in samples of microvesicles, cells and microvesicles were lysed in RIPA buffer (Cell signaling technology, Danvers, MA) and protein concentration estimated by the microBSA assay kit (Pierce, Rockford, IL). Approximately 20 micrograms of lysate were loaded in each lane and the membranes were probed overnight (1:1000) by either Rabbit anti-63 antibody (SBI Biosciences, Mountain View, CA), Rabbit anti-hsp70 (SBI Biosciences), rabbit STAT3 (Cell signaling technology), and/or rabbit phospho-STAT3 (Cell signaling technology).

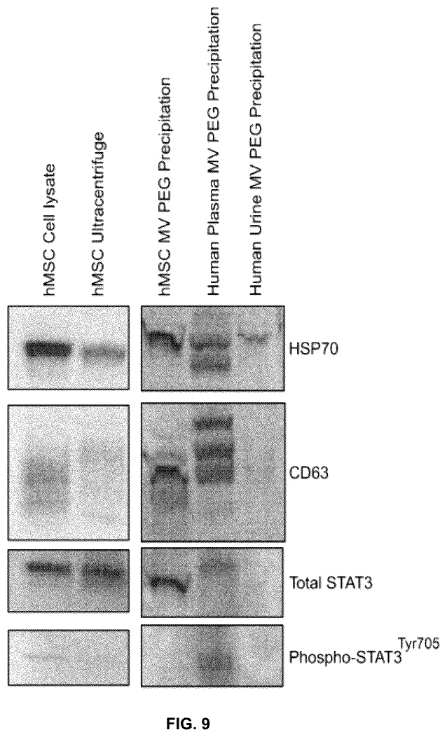

The presence of the exosomal markers (HSP 70 and CD63) confirmed that the methods of the present disclosure were capable of isolating exosomes. Further, the exosomes also contained the transcription factor STAT3 and the activated phosphorylated form phospho-STAT3. See FIG. 9.

### Example 8: Isolation of Microvesicles from Medium Conditioned Using a Culture of GFP-Labeled Bone Marrow-Derived Mesenchymal Stem Cells by the Methods of the Present Disclosure

Homozygous transgenic mice expressing the enhanced Green Fluorescent Protein (GFP) under the direction of the human ubiquitin C promoter (C57BL/6-Tg(UBC-GFP)30Scha/J) were obtained from Jackson Laboratories (Bar Harbor, Maine). These mice are known to express GFP in all tissues.

GFP-Mice (approximately 3-4 weeks of age) were euthanized by CO₂ asphyxiation. The limbs were cut above the hip and below the ankle joint. The hind limbs were harvested and skin, muscle, and all connective tissue was removed. The bones were then placed in a dish of ice cold sterile IX PBS and washed several times in PBS. The ends of each bone were snipped off with scissors. A 10 cc syringe with warmed medium (α-MEM supplemented with 20% fetal bovine serum and 1% penicillin/streptomycin/glutamine) was forced through the bone shaft to extract all bone marrow into a 150 mm plate. This was repeated several times to ensure all the marrow was removed. The cell mixture was pipetted several times to dissociate cells and the cell suspension was passed through a cell strainer (70 μm size) (BD Biosciences, San Jose, CA) to remove large cell clumps or bone particles.

Initial cultures were seeded between 2-3×10⁵ cells/cm² in tissue culture-treated dishes (BD Biosciences, San Jose, CA) and placed in a cell incubator at 37° C. in 95% humidified air and 5% CO₂. After 72-96 hours, the non-adherent cells were removed, the culture flasks were rinsed once with PBS, and fresh medium was added to the flask. The cells were grown until 80% confluence was reached and then passaged by Trypsin-EDTA (Life technologies, Carlsbad, CA). Cells were split at a 1:4 ratio.

Alternatively, cryopreserved GFP Mouse-MSC's were thawed at 37° C. and immediately cultured in α-MEM supplemented with 20% fetal bovine serum and 1% penicillin/streptomycin/glutamine at 37° C. in 95% humidified air and 5% CO₂. They were expanded similar to above.

The cells were grown in the flasks until 100% confluence was reached (approximately 1 week). The supernatant were transferred to 50 mL conical centrifuge tubes (Thermo Fisher Scientific Inc., Weston, FL) and immediately centrifuged at 400×g for 10 minutes at 4° C. to pellet any non-adherent cells. The supernatant was transferred to new 50 mL conical centrifuge tubes and centrifuged at 2000×g for 30 minutes at 4° C. to further remove cells and cell debris. The supernatants were collected and placed into 250 ml sterile, polypropylene disposable containers (Corning, Corning, NY). To the supernatant, RNase and protease free polyethylene glycol average molecular weight 6000 (Sigma Aldrich, Saint Louis, MO) at 8.5 w/v % and sodium chloride (final concentration 0.4 M) were added. The solution was placed in a cold room at 4° C. overnight with rocking. The solution was transferred to 50 mL conical centrifuge tubes and centrifuged at 10000×g at 4° C. for 30 minutes. The supernatant was decanted and the microvesicle enriched pellet resuspended in phosphate-buffered saline (PBS). The microvesicle enriched solution was transferred to Amicon ultra-15 centrifugal filter units (nominal molecular weight limit 100 kDa) (Millipore, Billerica, MA) and centrifuged at 5000×g for 30 minutes. The filter units were washed with phosphate-buffered saline and centrifuged again at 5000×g for 30 minutes. The concentrated sample was recovered (approximately 200-400 μl) from the bottom of the filter device. Protein concentration was determined by the micro BSA Protein assay kit (Pierce, Rockford, IL) and the enriched microvesicle solution was stored at −70 degrees or processed for downstream use (e.g. protein, RNA, and DNA extraction).

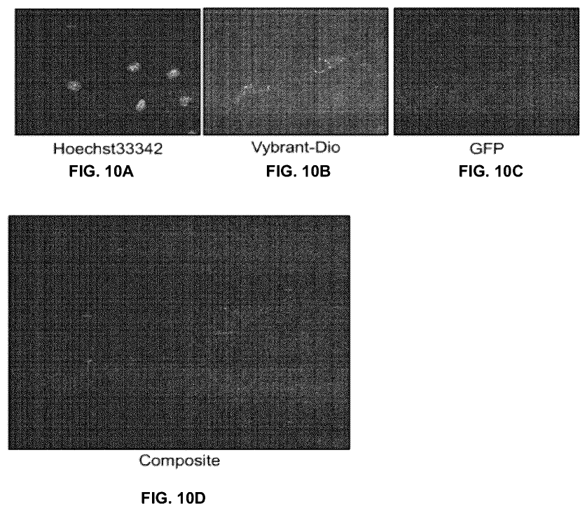

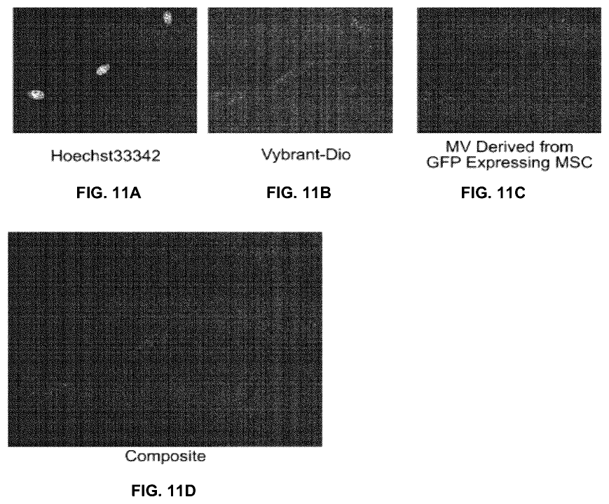

To determine cellular uptake of the microvesicles, normal human skin fibroblasts were labeled with Vybrant-Dio (Life technology) per manufacturer instructions. Normal skin fibroblasts were plated on fibronectin (Sigma-Aldrich) coated 4-well Nunc* Lab-Tek* II Chamber Slide (Thermo Fisher Scientific Inc.) (5×10 cells per well). Cells were stained with the nuclear dye Hoechst 33342 (Life technology) per manufacturer's instructions. Dil labeled fibroblasts were treated with microvesicles isolated from GFP expressing mouse MSC for 24 hours. Images were captured with an inverted IX81 Olympus microscope and ORCA-AG Hamamatsu digital camera. See FIG. 10A-D and FIG. 11A-D. Importantly, these images show that the microvesicles containing GFP were taken up by the cells.

### Example 9: EV Treatment of Acute GVHD in a Murine Model

In order to test the effect of EV treatment, a mouse model of GVHD was used. The EVs were administered daily before, not after inducing disease and continued daily for approximately 50 days. The model is a complete mismatched allogeneic model and without administering immunomodulation (i.e., EV treatment in advance), the recipients would all progress to lethal GVHD.

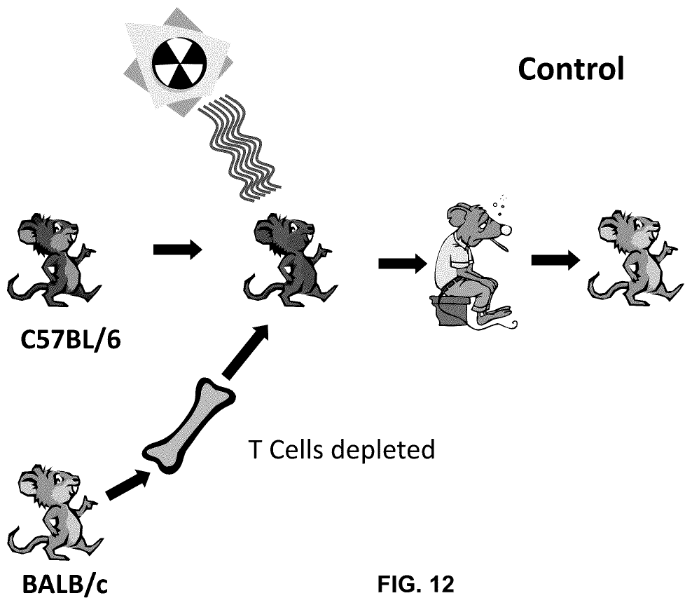

As illustrated in FIG. 12, a GVHD negative control experiment included using C57BL/6 mice that receive T cell depleted (TCD) bone marrow transplant only. These mice were symptomatic, but ultimately survived.

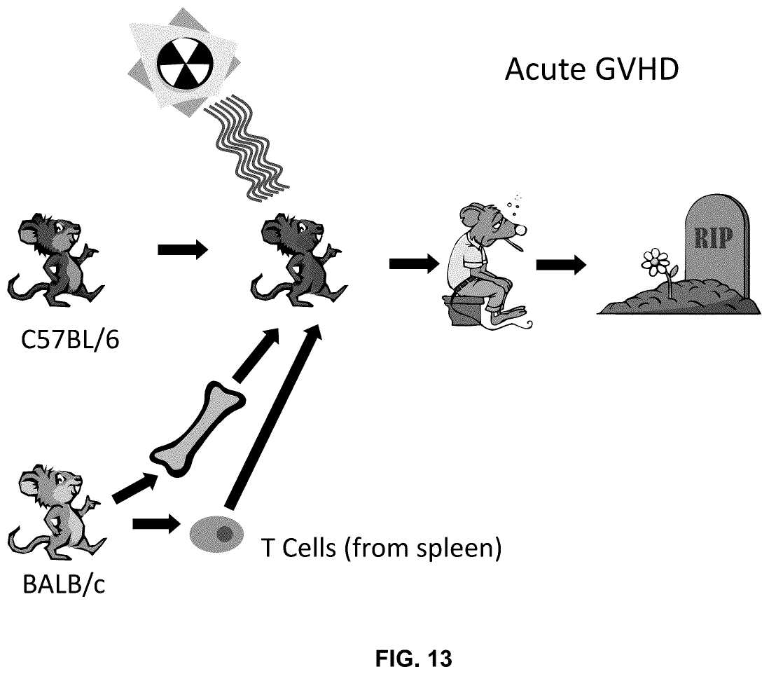

As illustrated in FIG. 13, an acute GVHD positive control experiment included using C57BL/6 mice without EV treatment. C57BL/6 mice received bone marrow and splenic T cells. As a complete MHC-mismatch model was used, these mice did not survive GVHD and typically 100% of these animals die within 28 days of transplant.

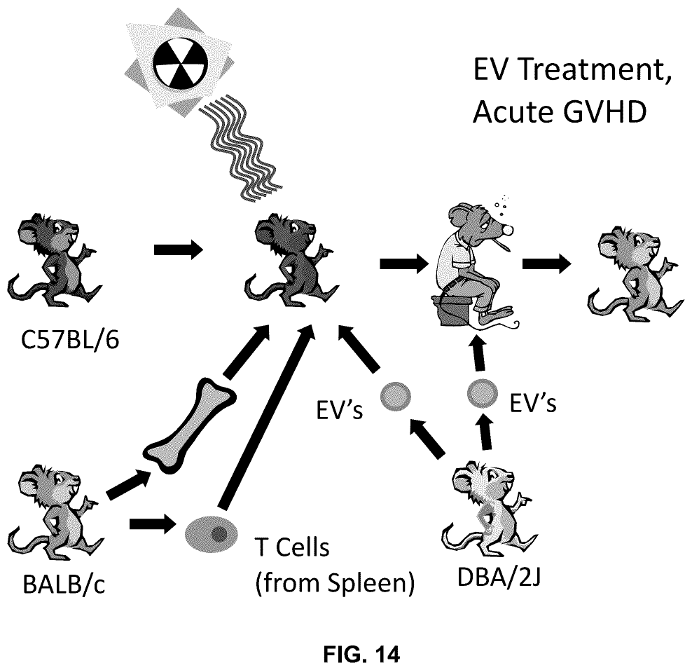

FIG. 14 illustrates the treatment group of acute GVHD model C57BL/6 mice and EV administration. C57BL/6 mice received bone marrow and splenic T cells. The mice also received EVs purified from the bone marrow of a DBA/2J mouse and go on to survive for a period of time until they were terminated due to the costs of prolonged housing. Of note, these mice did not succumb to their disease within the timeframe tested.

To carry out the four experiments described below, allogeneic hematopoietic cell transplantation (HCT) was modeled in BALB/C recipient mice (conditioned with 8 Gy total body irradiation) and murine histocompatibility complex (MHC) mismatched C57BL/6 recipients were infused with T cell-depleted bone marrow cells. C57BL/6 splenic T cells were co-infused on day 0 to induce acute graft versus host disease (GVHD). DBA/2 BM-MSC-EVs (EV) were administered by daily intraperitoneal injections (days 0-50) with dose and scheduling varying in successive experiments. Control recipients received saline injections rather than EV. Survival and clinical scores were assessed daily through day 120. T cell subsets from peripheral blood, spleen, and thymus were analyzed by flow cytometry on day 21 and day 28. Tissue was collected for histological analysis at day 28.

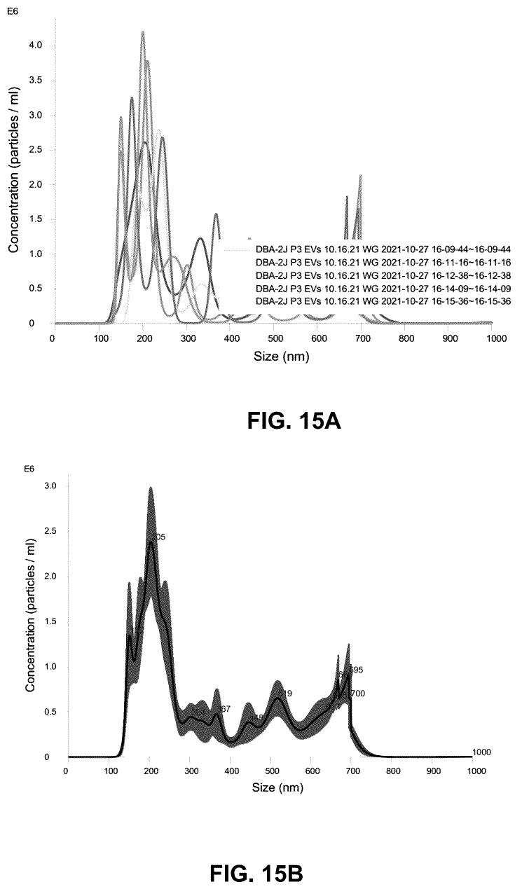

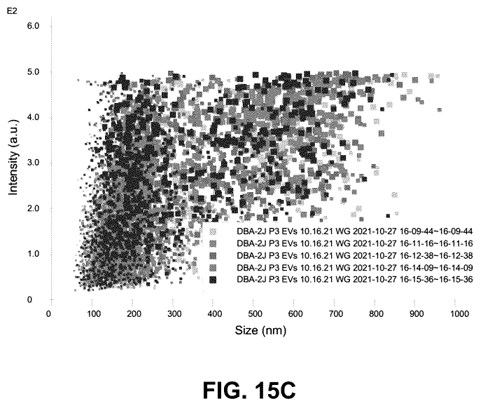

Four experiments were carried out testing 0.1×, 1×, 5×, and 10× doses of EVs. A 10× dose is equivalent to 16 μg of EVs. The 10× dose is also equivalent to about 0.8 mg/kg or 3.5×10¹⁰ particles/kg. In all experiments, experimental and control groups consisted of five mice. FIG. 15A-C depict NanoSight™ nanoparticle tracking analysis of the microvesicles and demonstrates that the majority of the microvesicles range in size from about 100 nm to 700 nm.

### Detailed Methods

### Hematopoietic Stem Cell Transplantation (HSCT)

For the HSCT in the major MHC-mismatch model (B6 to BALB/c), female BALB/c mice (H2d) received ablative conditioning with 8.0 Gy (Cs 137 source) body irradiation 1 day prior to transplant. BM cells were obtained from femurs, tibias, and vertebrae from sex-matched B6-CD45.1 (H2b; Thy1.2) donor animals. A single-cell suspension of marrow cells was prepared by flushing bones with a 21-gauge needle, and the cells were filtered through a 100 μm nylon mesh. T cell depletion (TCD) of donor marrow cells was achieved via complement-mediated lysis using anti-T cell-specific antibody HO134 (hybridoma supernatant, mouse anti-Thy1.2 IgM, ATCC®) and rabbit complement (Cedarlane Laboratories®.) The marrow cells were incubated at 37° C. for 45 minutes, washed twice in RPMI, and resuspended for hematopoietic cell transplant. Marrow TCD was routinely >99%. Donor T cells were prepared from spleens obtained from B6 mice. Donor T cells were stained with anti-CD4, clone RM4-5; anti-CD8, clone 53-6-7 and adjusted to 1.0×106 T cells per mouse prior to mixing with BM. Recipient mice were transplanted (day 0) with TCD BM (5×106) and 1.0×106 T cells through intravenous administration in a 0.2-ml volume via tail vein injection. DBA/2 bone marrow (BM)-MSC derived extracellular vesicles (EV) or saline were administered by daily intraperitoneal injections (days 0-50) at (10×) 16 μg and (5×) 8 μg per injection in 200 μl for experiment 3 and (10×)16 μg for experiment 4. For experiment 1, EV or saline were given once per week at (1×)1.6 and (0.1×) 0.16 μg per injection in 200 μl for 40 days. For experiment 2, EV or saline were given daily at 1.6 and 0.16 μg per injection in 200 μl for 40 days.

### GVHD Scoring

GVHD was assessed using the scoring approach developed by Cooke et al. by monitoring recipients for changes in total body weight, clinical signs, and overall survival (Cooke et al. “An experimental model of idiopathic pneumonia syndrome after bone marrow transplantation” Blood 1996 Oct. 15; 88(8):3230-9.) The clinical signs of GVHD were recorded for individual mice. Recipients were scored on a scale from 0 to 2 for 5 clinical parameters: (a) weight loss; (b) diarrhea; (c) fur texture; (d) posture; and (e) alopecia.

### Overview of the Design of Experiments 1-4

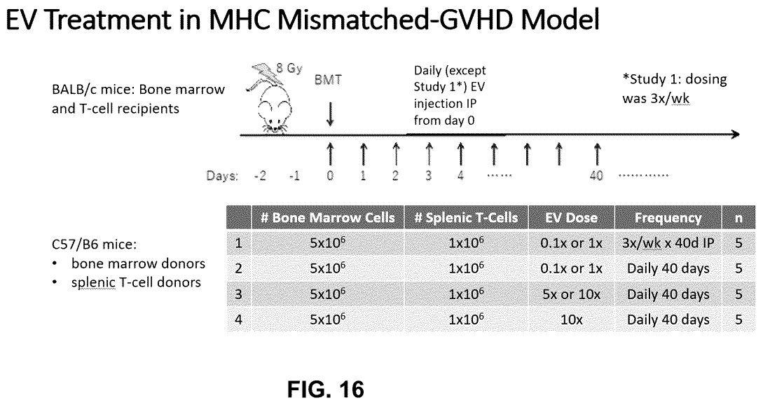

A schema of experiments 1-4 is shown in FIG. 16.

An overview of the design of experiments 1-4 is shown in Table 1 below.

TABLE 1
Design of Experiments 1-4

| Experiment 1 | Experiment 2 | Experiment 3 | Experiment 4 |
|---|---|---|---|
| Low dose | Low dose | Comparison of 5× vs. | Repeat with 10× |
| (0.1× and 1×** ) | (0.1× and 1×) | 10× dose | dose |
| 3×/week for 40 days | Daily for 40 days | Daily for 50 days | Daily for 50 days |
| n = 3 mice per group | n = 5 mice per group | n = 5 mice per group | n = 5 mice per group |

**1× dose an estimate from preclinical topical experiments on burns and wounds

### First Experiment

In the first experiment, a low dose (0.1× or 1×) of EVs was used in the treatment groups. The 1× dose was based on preclinical topical experiments on burns and wounds. The EVs were administered at 1.6 μg/200 μl (1× dose) or 0.16 μg/200 μl (0.1× dose) by intraperitoneal injection three times per week for 40 days.

Four groups of animals were studied: group 1 (bone marrow alone as a negative control for GVHD), group 2 (bone marrow and donor T cells as a positive control for lethal GVHD), group 3 (GVHD model and 1× dose of EVs); group 4 (GVHD model and 0.1× dose of EVs).

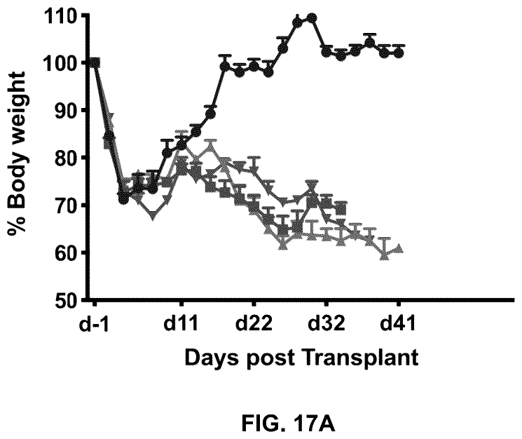

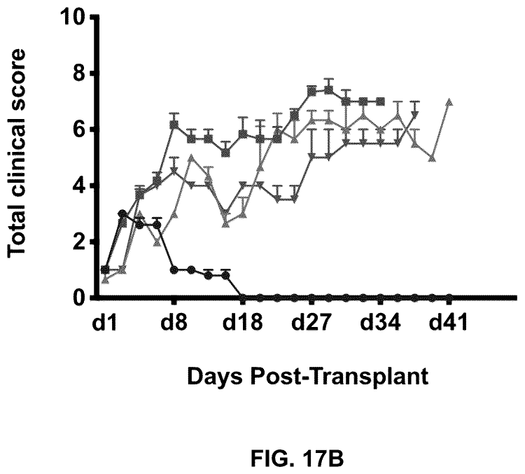

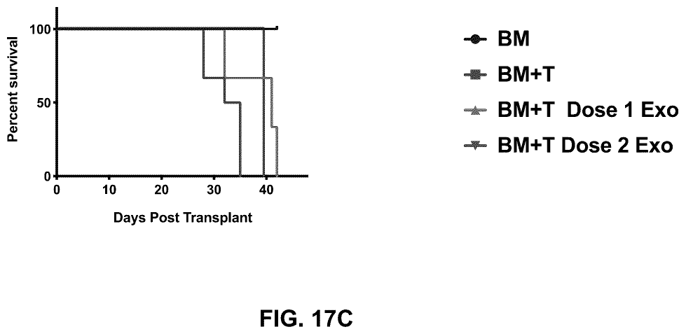

As shown in FIG. 17, EVs administrated three times per week (at both the 1× and 0.1× doses) slightly decreased overall GVHD clinical scoring (FIG. 17B) and slightly prolonged survival in mismatched murine HCT (FIG. 17C) but was insufficient to cure mice of lethal GVHD. As a complete WIC-mismatch model designed for 100% non-survival was used, as anticipated all GVHD model mice that were not treated with EVs were dead within about 28 days.

### Second Experiment

In the second experiment, a low dose (0.1× or 1×) of EVs was used in the treatment groups, as in experiment 1. Experiment 2 differed from experiment 1 in that EVs were administered daily for 40 days.

The results were a significant improvement in survival in EV treated mice with survival to 40 days. All GVHD model mice that were not treated with EVs were dead within about 28 days.

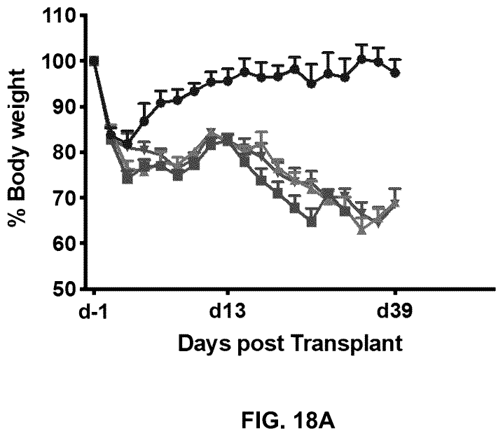

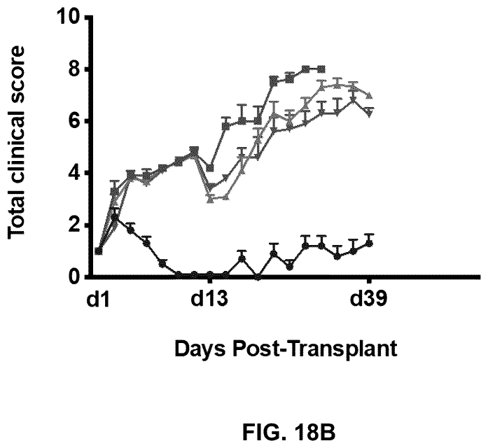

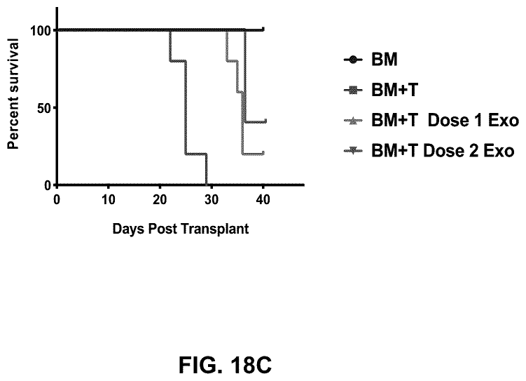

As shown in FIG. 18, daily dosing of EVs (at both the lx and 0.1× doses) slightly decreased overall GVHD clinical scoring (FIG. 18B) and significantly prolonged survival (FIG. 18C) in mismatched murine HCT but was insufficient to cure mice of lethal GVHD. This experiment demonstrated that increased frequency of administration in the murine model (daily versus 3 times per week) improved efficacy of the EV treatment, demonstrating a schedule dependence of EV dosing.

### Third Experiment

In the third experiment, a 5× and a 10× dose of EVs were used in the treatment groups. The EVs were administered daily for 40 days. The EVs were administered at 8 μg/200 μl (5× dose) or 16 μg/200 μl (10× dose) by intraperitoneal injection. Four groups of animals were studied: group 1 (bone marrow alone as a negative control for GVHD), group 2 (bone marrow and donor T cells as a positive control for lethal GVHD), group 3 (GVHD model and 5× dose of EVs); group 4 (GVHD model and 10× dose of EVs).

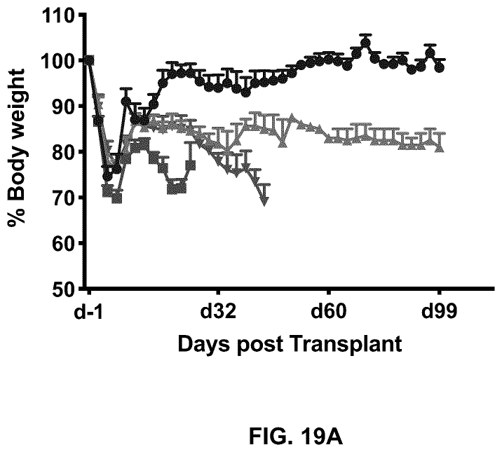

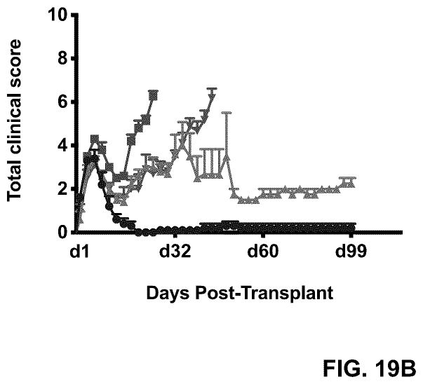

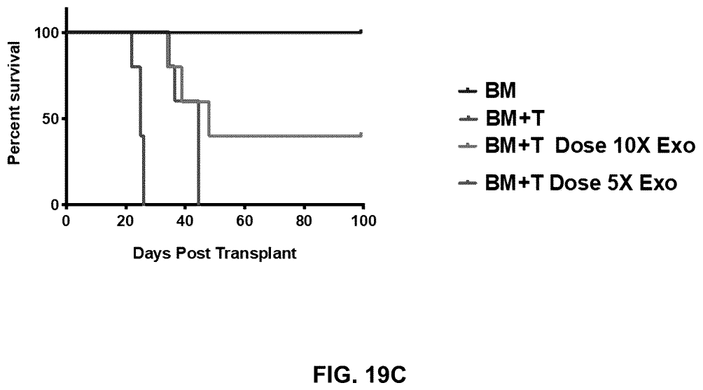

As shown in FIG. 19, daily dosing of the EVs at the higher 5× and 10× doses significantly improved overall GVHD clinical scoring (FIG. 19B) and prolonged survival (FIG. 19C) in mismatched murine HCT. Clinical scores that were much better in the 10× group. Additionally, 10× dosing was able to rescue a subset of mice from lethal GVHD, with effects that persisted after discontinuation of dosing at 40 days post-transplantation. All GVHD model mice that were not treated with EVs were dead within about 28 days. As shown in FIG. 19C, all EV treated mice survived to the last day of treatment.

As shown in FIG. 19A, the non-GVHD control mice had the highest body weight followed by the mice in the 10×EV treatment group. Mice in the 5× dose treatment group were sacrificed at the end of treatment due to weight loss (these mice were alive but sacrificed due to University of Miami veterinary recommendations.) There was 50% survival at 100 days for the 10× dose EV treatment group (50 days after discontinuing treatment.) Clinical scores remained stable after 50 days for both non-GVHD control and EV treated mice.

This experiment further demonstrated the dose and schedule dependency of EV treatment and now the ability to prevent lethality of GVHD.

### Fourth Experiment

In the fourth experiment, a 10× dose of EVs was retested. The EVs were administered daily for 40 days. The EVs were administered in an intraperitoneal injection of 16 μg/200 μl (10× dose). Three groups of animals were studied: group 1 (bone marrow alone as a negative control for GVHD), group 2 (bone marrow and donor T cells as a positive control for lethal GVHD), and group 3 (GVHD model and 10× dose of EVs).

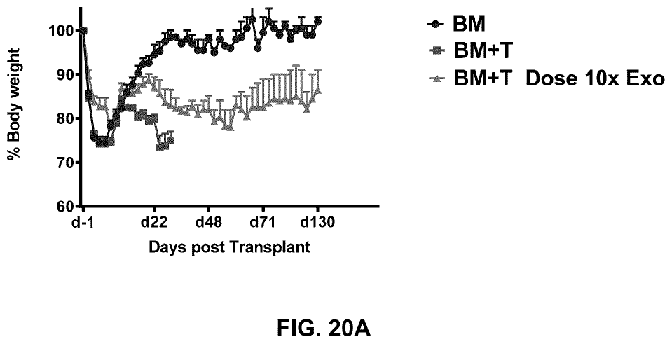

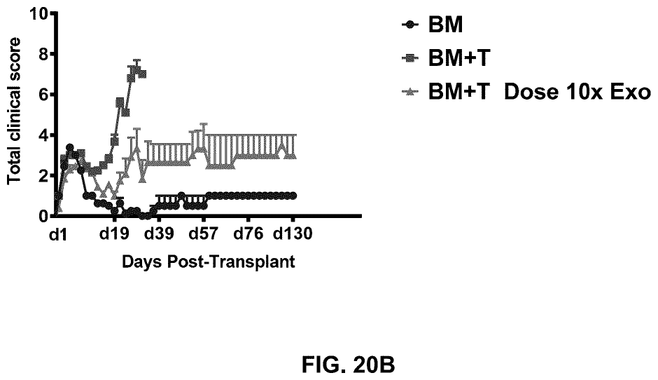

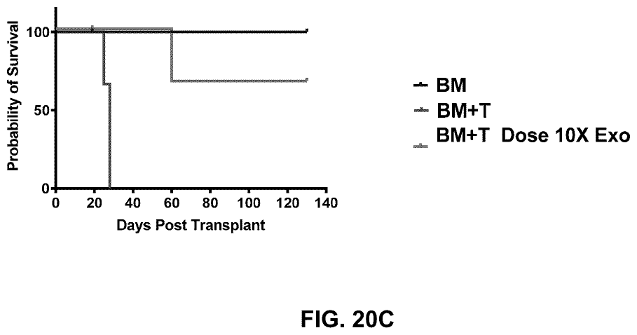

As shown in FIG. 20, the results were that daily dosing of EVs at the 10× dose significantly improved overall GVHD clinical scoring (FIG. 20B) and significantly prolonged survival (FIG. 20C) in mismatched murine HCT. EV treatment was able to rescue a majority of treated mice from lethal GVHD, with effects that persisted after discontinuation of dosing at 40 days post-transplantation. There was 75% survival at 140 days in the treatment group (90 days after discontinuing the treatment.) The surviving animals at 140 days were sacrificed due to the costs of prolonged housing. Of note, these mice did not succumb to their disease within the timeframe tested. Clinical scores remained stable for 100 days for both non-GVHD control and EV treated mice. All GVHD model mice that were not treated with EVs were dead within about 28 days. This example further demonstrated the dose and schedule dependency of EV dosing and the ability to prevent lethality of GVHD.

On overview of the results of Experiments 1˜4 is shown in Table 2 below.

TABLE 2
Results from Experiments 1-4

| Experiment 1 | Experiment 2 | Experiment 3 | Experiment 4 |
|---|---|---|---|
| Clinical score | Significant clinical | Significant clinical | Significant clinical |
| improvement and | score improvement | score improvement in | score improvement in |
| increased survival in | with survival to 40 | treated group-10× >> | treated group (stable |
| treated group; most | days in treated group | 5× (stable at 50 days, | at 100 days, like |
| received all doses | All GVHD untreated | like control); >50% | control); >75% |
| All GVHD untreated | dead by 28 days | survival at 100 days^{$} | survival at 140 days^{#} |
| dead by 28 days | | in treated group | in treated group |
| | | All GVHD untreated | All GVHD untreated |
| | | dead by 28 days | dead by 28 days |

^{$}50 days after discontinuing treatment
^{#}90 days after discontinuing treatment

### Conclusions and Correlative Studies

In conclusion, EVs were shown to be effective in a GVHD mouse model and showed a dose response. Other effects observed were long term survival or treatment benefit after treatment has stopped. Furthermore, there were no adverse events with high dose and daily administration. Additionally, the EV equivalent dose was higher than what can be given by MSCs (i.e. cells). The dose used would equate to 12.5 billion MSCs per day in a human.

Correlative studies demonstrated dramatic improvements in skin and gut pathology, decreased destruction of primary and secondary lymphoid organs (lymph nodes, thymus) and improved T cell subset and thymus reconstitution after EV treatment, consistent with profound and persistent induction of tolerance.

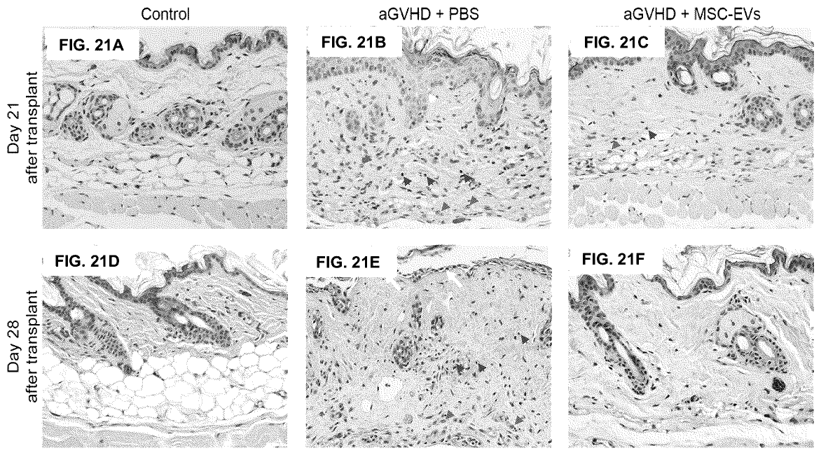

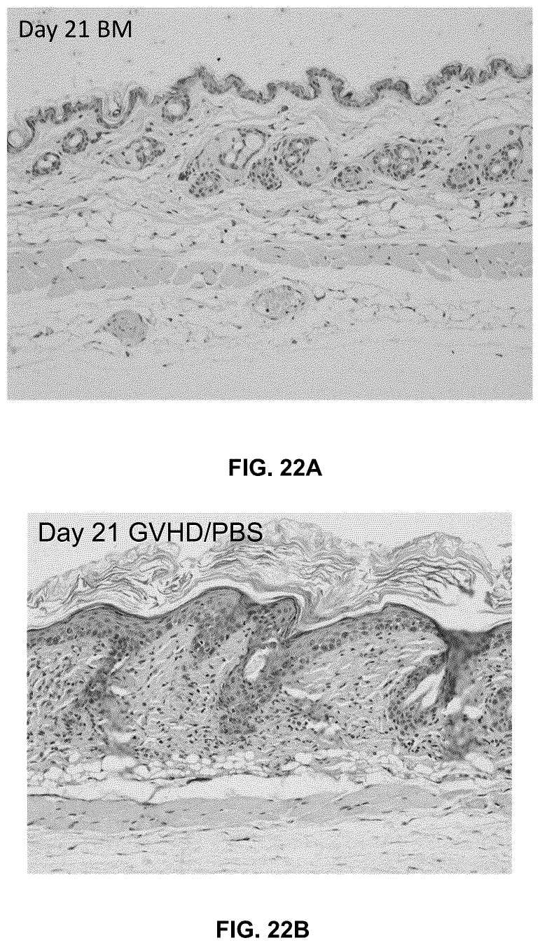

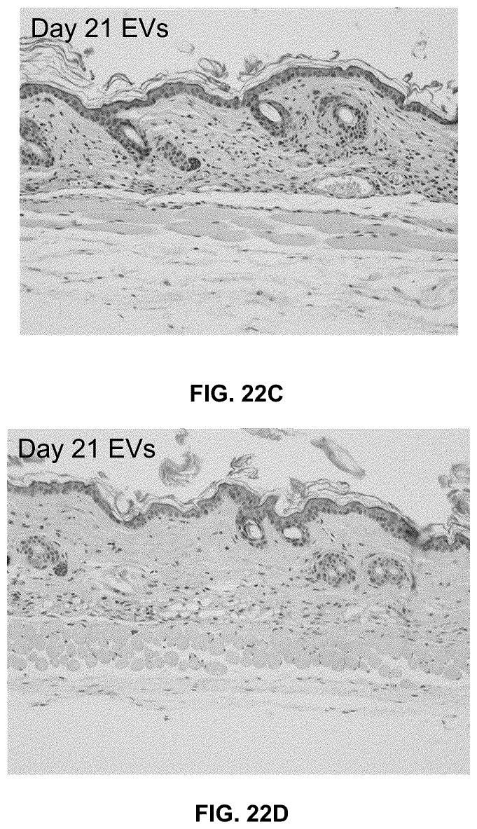

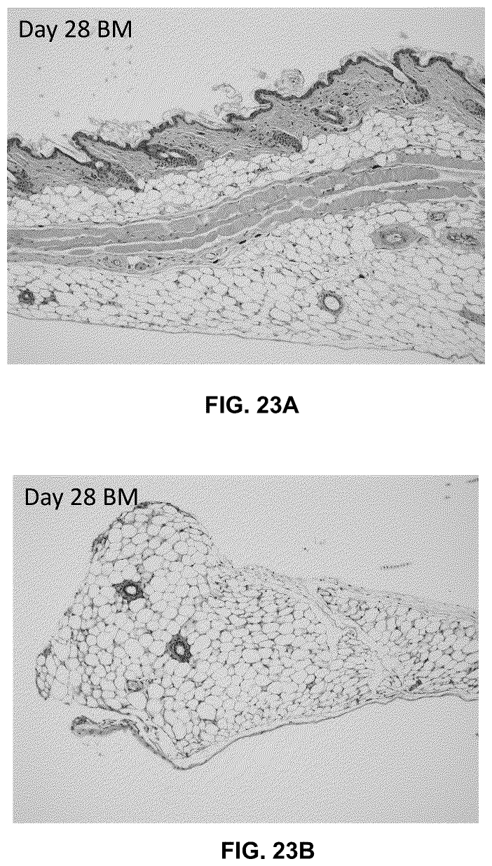

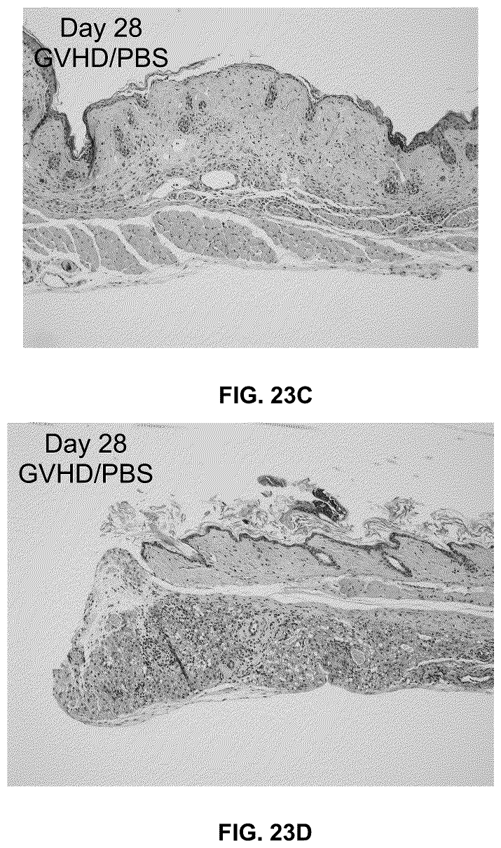

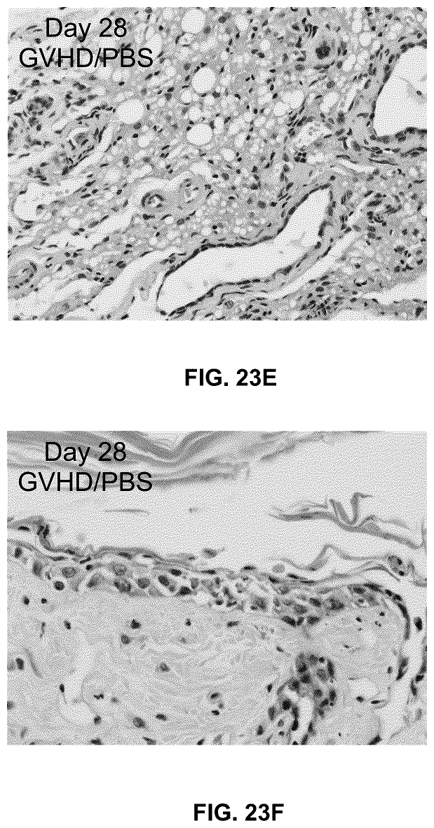

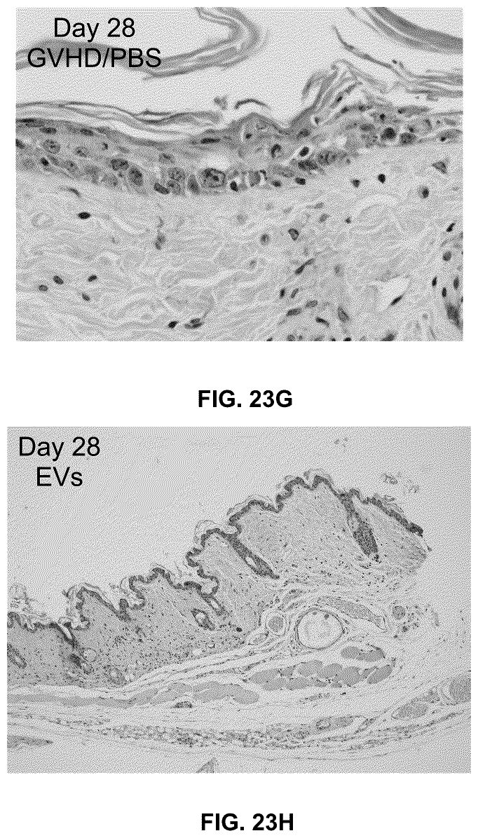

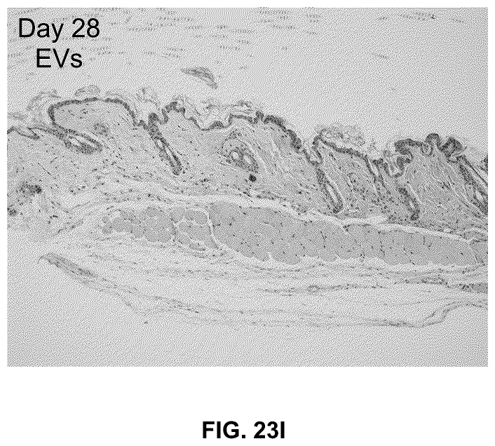

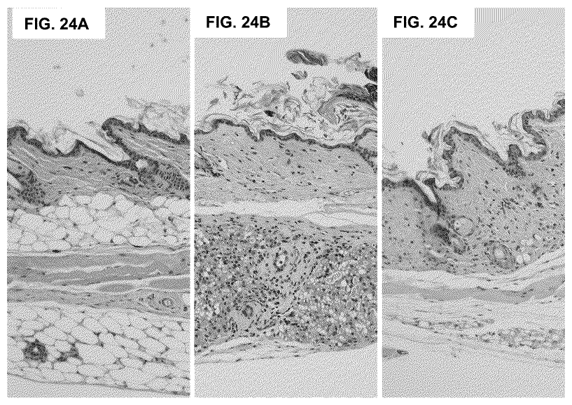

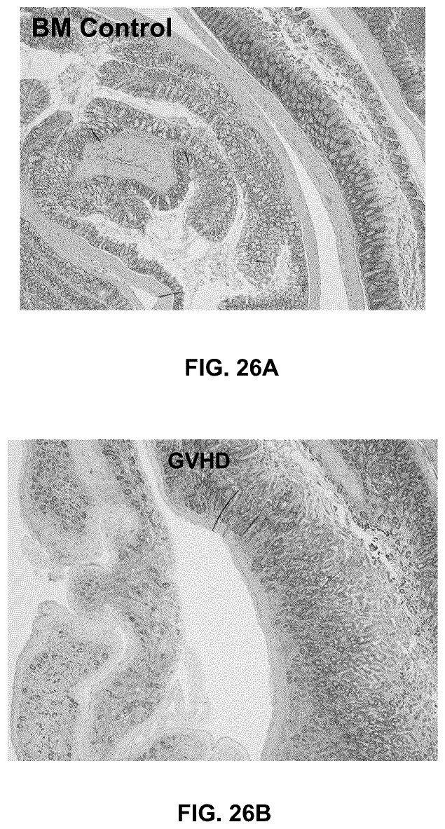

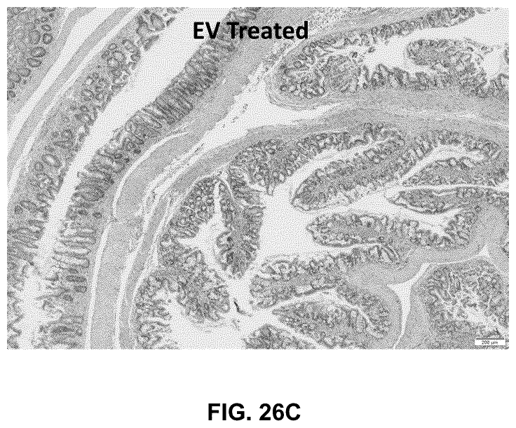

As shown in FIG. 21A-F, FIG. 22A-D, and FIG. 23A-I MSC-EVs attenuate cutaneous acute GVHD pathology. The red arrowheads in FIG. 21 point to lymphocytes and the green arrowheads point to histiocytes, both of which are increased in the GVHD model mouse but decreased with EV treatment. Furthermore, as shown in FIG. 24A-C, MSC-EVs attenuate subcutaneous inflammation in aGVHD pathology. FIG. 26A-C demonstrates that microvesicle treatment attenuate colon inflammation. These results show that EV treatment decreases tissue manifestations of GVHD.

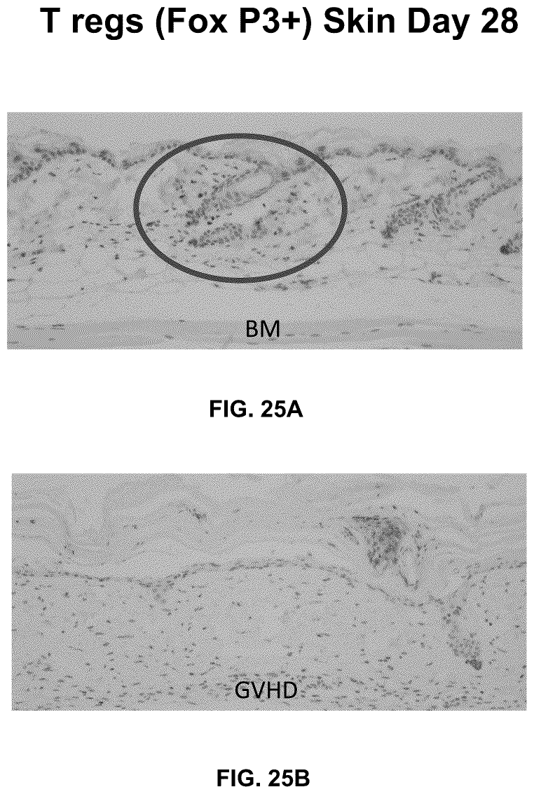

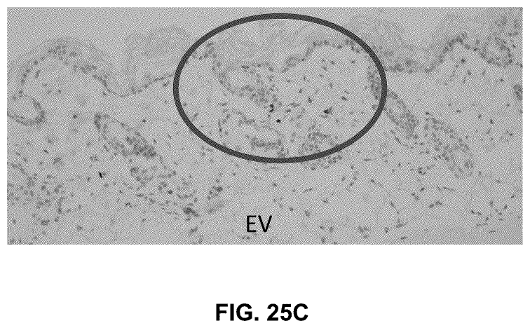

FIG. 25A-C demonstrate that microvesicle treatment increases FOXP3+ regulatory T (Treg) cells in skin tissue samples from mice at 28 days after transplant. These results show that FOXP3+ regulatory T (Treg) cells in skin tissue that are lost in the GVHD model are restored with EV treatment.

Furthermore, the GVHD pathology changes seen in the GVHD model mouse are similar to those seen in this acute phase of chronic GVHD. Therefore, the ability of EVs to attenuate these changes may provide important insight into how the EVs can be effective in the chronic (later) phases of GVHD.

1. A method of preventing or treating graft versus host disease (GVHD) in a subject comprising administering to the subject a pharmaceutical composition comprising intact microvesicles isolated from a biological fluid of an unrelated or related donor using polyethylene glycol (PEG) precipitation, wherein one or more symptoms of GVHD comprising weight loss, cutaneous tissue damage, subcutaneous tissue damage, cutaneous inflammation, satellite cell necrosis, truncated lifespan, and/or subcutaneous inflammation are prevented or alleviated in the subject.

2. The method of claim 1, wherein the subject has received a hematopoietic stem cell transplant from a person wherein the donor is matched, partially mismatched, or completely mismatched,
optionally wherein the hematopoietic stem cells can be sourced from bone marrow, peripheral blood and/or umbilical cord blood, which is freshly collected and/or cryopreserved then infused.

3. The method of claim 1, wherein the method further comprises administering an additional therapeutic agent to the subject.

4. The method of claim 3, wherein the additional therapeutic agent is selected from the group consisting of a steroid, anti-metabolite, calcineurin inhibitor, mTOR inhibitor, kinase inhibitor, signal transducer and activator of transcription (STAT) inhibitor, and nucleotide analog inhibitor,
optionally wherein the additional therapeutic agent or process is selected from the group consisting of tacrolimus, monoclonal and/or polyclonal antibodies including antithmyocyte, globulin, abatacept, sirolimus, post-transplant cyclophosphamide, itacitinib, ibrutinib, belumosudil, and extracorporeal photopheresis.

5. (canceled)

6. The method of claim 1, wherein the PEG comprises a weight of about 6000-20000 Da.

7. The method of claim 1, wherein the intact microvesicles comprise one or more of the following: exosomes, apoptotic bodies, ectosomes, nanovesicles, microparticles, membrane particles, extracellular vesicles, and shedding vesicles.

8. The method of claim 1, wherein administration of the pharmaceutical composition is terminated and the subject survives for a period of time without further administration of the pharmaceutical composition wherein the period of time where the subject survives is more than ninety days after discontinuing treatment.

9. (canceled)

10. The method of claim 1, wherein the biological fluid comprises mesenchymal stem cells derived from bone marrow.

11. (canceled)

12. The method of claim 10, wherein the pharmaceutical composition is administered to the subject either at the time of transplant, before the transplant, after the transplant, or a combination thereof.

13. (canceled)

14. The method of claim 1, wherein the subject has acute GVHD or chronic GVHD.

15. The method of claim 1, wherein the intact microvesicles are purified by tangential flow filtration.

16. The method of claim 1, wherein the intact microvesicles range in size from 2 . nm to 5000 nm.

17. (canceled)

18. The method of claim 1, wherein the intact microvesicles have a molecular weight of at least 100 kDa.

19. (canceled)

20. The method of claim 1, wherein the GVHD is refractory to a treatment selected from the group consisting of a steroid, anti-metabolite, calcineurin inhibitor, mTOR inhibitor, kinase inhibitor, signal transducer and activator of transcription (STAT) inhibitor, and nucleotide analog inhibitor.

21. The method of claim 1, wherein the intact microvesicles deliver one or more bioactive agents comprising check-point inhibitors, transcription factors, peptides, subcellular organelles, and/or nucleic acids to the subject.

22. The method of claim 1, wherein the administration of intact microvesicles increases the number of regulatory T cells (Tregs) in tissue of the subject as compared to a subject who was not administered the pharmaceutical composition.

23. The method of claim 22, wherein the Tregs are FOXP3+.

24. The method of claim 1, wherein the intact microvesicles are delivered to the subject by systemic administration, local injection, and/or topically to skin or eye.

25. The method of claim 1, wherein the intact microvesicles are delivered intravenously to the subject.

26. A pharmaceutical composition for use in preventing or treating graft versus host disease (GVHD) in a subject comprising intact microvesicles isolated from a biological fluid using polyethylene glycol (PEG) precipitation, wherein administration of the pharmaceutical composition alleviates or prevents one or more symptoms of GVHD in the subject,
wherein the one or more symptoms of GVHD comprise weight loss, cutaneous tissue damage, subcutaneous tissue damage, cutaneous inflammation, satellite cell necrosis, truncated lifespan, and/or subcutaneous inflammation.

27-50. (canceled)
